(19) United States
(12) Patent Application Publication
(10) Pub. No.: US 2022/0064595 A1
(43) Pub. Date: Mar. 3, 2022

(54) MULTI-TARGET CHIMERIC ANTIGEN RECEPTOR

(71) Applicant: Shenzhen Beike Biotechnology Co., Ltd., Shenzhen (CN)

(72) Inventors: Lei WANG, Tianjin (CN); Bin GAO, Tianjin (CN); Yasong WU, Tianjin (CN); Yuan SI, Tianjin (CN); Dan LV, Tianjin (CN); Qing WEI, Tianjin (CN)

(21) Appl. No.: 16/475,030

(22) PCT Filed: Dec. 27, 2017
(86) PCT No.: PCT/CN2017/118980

§ 371 Date: Jun. 28, 2019

(30) Foreign Application Priority Data
Dec. 29, 2016 (CN) 201611246563.4

(51) Int. Cl. C12N 5/0783; C12N 15/63; C07K 14/725; C07K 14/715

(52) U.S. Cl. CPC C12N 5/0636; C12N 15/63; C07K 14/715; C07K 14/7051; C12N 5/0646

(57) ABSTRACT

Disclosed in the present invention is a multi-target chimeric antigen receptor. Provided in the present invention is a multi-target chimeric antigen receptor consisting of a main peptide chain and an auxiliary peptide chain; the main peptide chain comprises an antigen binding domain A, an auxiliary peptide chain connecting domain B, a transmembrane domain C and an intracellular signaling domain D; the auxiliary peptide chain comprises a main peptide chain connecting domain F; the antigen binding domain A is a polypeptide having an antigen-binding function; the auxiliary chain connecting domain B and the main peptide connecting domain F are combined with each other; the transmembrane domain C is a transmembrane domain of any membrane-binding protein or a transmembrane protein; and the intracellular signaling domain D comprises a primary signaling region. The multi-target chimeric antigen receptor of the present invention can bind to different antigens through the two antigen binding domains thereof, and mediates specific cell killing; and a cytokine and cytokine receptor complex playing the role of a cytokine are introduced into the multi-target chimeric antigen receptor of the present invention.

### RELATED APPLICATIONS

The present application is a National Phase of International Application Number PCT/CN2017/118980, filed Dec. 27, 2017, and claims the priority of China Application No. 201611246563.4, filed Dec. 29, 2016.

### INCORPORATION BY REFERENCE

The sequence listing provided in the file entitled

C6351-007_Sequence_listing_v2.txt,

which is an ASCII text file that was created on Aug. 28, 2020, and which comprises 105,249 bytes, is hereby incorporated by reference in its entirety.

### TECHNICAL FIELD

The present invention relates to the field of biotechnology, and, in particular, to a multi-targeting chimeric antigen receptor.

### BACKGROUND

The chimeric antigen receptor (CAR) is usually a transmembrane fusion protein consisting mainly of an antigen binding domain, a transmembrane domain and an intracellular signaling domain (patent CN 105392888 A). Immune cells (e.g., T cells or NK cells) will be conferred antigen specificity upon expression of a chimeric antigen receptor.

The antigen-binding domain of the CAR molecule determines the specific targeting of immune cells. A typical CAR often uses a single-chain variable fragment (scFv) as its antigen-binding domain consisting of the variable region of the heavy chain of the antibody and the light chain variable region joined by a linker. Because of its simple structure, there is only one peptide chain, which is suitable for integration into the CAR molecule as part of the fusion protein. When the tumor antigen itself is a receptor, the antigen binding domain of the CAR can also be replaced by its ligand specific for the receptor.

The second unit structure of the CAR molecule is the transmembrane (TM), which allows CAR molecules to be expressed on the cell surface across the membrane. Usually CAR molecules are armed on T cells for tumor treatment, therefore, many transmembrane regions of T cell-expressing proteins, such as CD3ζ, CD4, CD8 or CD28 can be used as CAR transmembrane domain. It has been reported in the literature that the expression level of CAR with the transmembrane region of CD28 is higher than that with the transmembrane region of CD3ζ. In addition, in some CAR molecular structures, a spacer/hinge region can be inserted between the antigen domain and the transmembrane domain, which can reduce the steric hindrance of the antigen structure and the targeted antigen. Fragmental peptides of IgG1, IgG4, IgD and CD8 are often used for such purpose.

The intracellular signaling region is the most important part of the CAR molecule. It is responsible for transmitting intracellular signals and the activation and proliferation of immune cells. The early design of the intracellular signaling domain used one CD3ζ stimulation signal only, also known as the first generation CAR. The same as the CD3 ζ molecule, there are only 3 ITAMs without the second signal of co-stimulation. Therefore, the first generation CAR has only weak anti-tumor effect since T cell is difficult to proliferate due to lack of the second signal when T cells bind to the antigen of the tumor, so it is less effective in clinical applications. On this basis, CAR was modified to integrate the active domain of the CD28 molecule that transmits the second signal in the CAR, hence called as the second generation CAR. Addition of co-stimulatory signals to CAR-T cells can enhance the survival of CAR-T cells in vivo and enable cells to kill their tumor cells while maintain proliferation, improving the anti-tumor capacity of CAR-T cells. Therefore, the second-generation CAR showed good anti-tumor effects in clinical applications. For example, Alvavez-Vallina and Hawkins et al. inserted the costimulatory molecule CD28 signal fragment into the CAR molecule, demonstrating that T cells can be stimulated by the corresponding antigen (Alvarez-Vallina L, Hawkins R E., 1996). Therefore, the second generation CAR showed a good anti-tumor effect in clinical applications. However, in the absence of exogenous co-stimulatory molecules, all second generations of CAR could not produce sufficient IL-2 to promote T cell proliferation. However, if transgenic B7 costimulatory molecules were given, these transgenic CAR-T cells could produce IL- 2 to promote proliferation themselves. Based on this, a third-generation CAR was generated, in which the intracellular signal region has another co-stimulatory molecule in addition to the CD28 molecule, usually OX40 or 4-1BB (Till BG, Jensen M C, Wang J, et al., 2012). Compared with the 2nd generation CAR, the 3rd generation CAR has considerable cytotoxicity while its ability to expand in vivo and produce cytokines is better (Pule, M. A., Straathof, K. C., Dotti, G., et al., 2005). However, recent studies have shown that the functional activity of CAR-T cells is related to the intracellular receptors provided by the target cells in addition to their intracellular signal sequences. Whether the third-generation CARs are superior to the second-generation CARs remains to be clinically validated.

Chimeric antigen receptor T cell therapy has achieved good results in B-cell-derived tumors. The clinical outcome of the treatment with murine antibody based CART19 (“CTL019”) has shown promising results in the establishment of complete response for CLL patients and children ALL. Relief (Kalos, M., Levine, B. L., Porter, D. L., et al., 2011; Porter et al., 2011; Grupp, et al., 2013), opened up a new era of adoptive treatment of cancer.

In vitro expansion of chimeric antigen receptor-immune cell therapy T cells or NK cells relies on IL-2 stimulation, so a certain amount of IL-2 must be added to the T and NK cell culture media (Bodnar et al, 2008; Grund et al., 2005). In order to simplify the culture of NK cell and in view of the side effects of toxicity with IL-2 in clinical applications, researchers have genetically engineered IL-2 dependent NK92 cells to stably express IL-2, which makes the addition of IL-2 in the culture medium unnecessary (Shigeki Nagashima, 1998; YK TAM, 1999). IL-15 is functionally similar to IL-2 and shares the same beta and gamma receptor unit. Studies indicate that IL-2 or IL-15 is required for survival and proliferation of NK cells and CD8+ T cells (Boyman et al., 2007). Therefore, the researchers constructed an IL-15-expressing NK cell line and found that IL-15-expressing NK92 cells could greatly reduce the amount of IL-2 required in the medium (JIAN ZHANG, 2004; Jiang et al., 2008; 03152968.2). Although IL-15 and IL-2 share the same fly receptor units, but each has a specific a receptor, it was found that IL-15Ra-sushi (the sushi domain of IL-15 receptor α) is a super-agonist in IL-15. Agonists can greatly enhance the function of IL-15 (Han et al., 2011; Mortier et al., 2006) (patent: 201280037114.2; 201510358540.1), and the complex of IL-15 and IL-15Rα-sushi can be completely used instead of the role of IL-2 in supporting the growth of T/NK cells (Peter S. Kim 1, 2016; Rosario et al., 2016), NK/CD8+T cells are activated and their cytotoxicity to kill tumors is increased. Therefore, there is a trend towards using the fusion of IL-15 and IL-15Rα-sushi complex or other functional cytokine and receptor complex in CAR construct to improve the efficacy of cellular immunotherapy.

At the same time, tumors are treated by a single target, tumor cells will escape by down-regulating the expression level of this target, and it will alleviate this problem to treat tumor by two targets (patent: 201710005395.8; 201510733585.2; 201710640609); 201610353118.1). A researcher, for example, can use a linker to connect sequences of two different antibodies or antigen ligands together as an extracellular portion of a chimeric antigen receptor, allowing Car-T cells to recognize two targets, which stops the decline in efficacy due to tumor immune escape (Hegde, M., Mukherjee, M., Grada, Z., et al., 2016; Schneider, D., Xion, Y., Wu, D., et al., 2017). Furthermore, researchers used an antibody to a biomarker named 5B9 tag as the extracellular domain of CAR-T, and a group of antibodies targeting different tumor targets were fused to the 5B9 tag. Car-T could obtainability to recognize different targets by the binding of different antibodies to respective antigens (Cartellieri, M., Feldmann, A., Koristka, S., et al., 2016).

In addition, if one of the chimeric antigen receptors is made to be an Immune checkpoint-related site, the activity of the immune cells can be greatly enhanced by blocking the negative signal transmitted by the immunological checkpoint. For example, PD-1 (programmed death 1) and its receptor PD-L1, PD-L2 are important regulators of T cell activity (Okazaki and Honjo, 2007). The binding of PD-1 on the surface of T cells to PD-L1/2 on other cells causes inhibition of T cells, which plays an important role in the process of avoiding autoimmune diseases and producing immune tolerance in humans, but some cells infected by pathogenic microorganisms and cancer cells can also evade T cell surveillance by up-regulating their PD-L1/2 on then or PD-1 expression on T-cell, leading to disease (Freeman et al., 2000; Keir et al., 2008; Parry et al., 2005).). Therefore, the researchers explored antibodies against PD-1 or PD-Ll to bind antigen to block this signaling pathway, which can significantly improve T cell activity and enhance the body's resistance to pathogenic microorganisms and cancer (Topalian et al., 2012; Yanan Guol, 2016). A combination of CAR-T therapy targeting MSLN with PD1 antibodies has shown that this combination increases the ability of CAR-T cells to kill tumor cells (Cherkassky, L., Morello, A., Villenavargas, J., et al., 2016). Furthermore, researchers have constructed a so-called chimeric antigen receptor switching system by linking immune checkpoint molecules PD1 or CTLA-4 to the intracellular signal region. In this way, the negative signal through immune checkpoints from tumor cells and tumor microenvironment was changed to increase the efficiency of CAR-T cells in treating tumors and reducing the risk of off-target effects (Liu, X., Ranganathan, R., Jiang, S., et al., 2016; Fedorov, V. D., Themeli, M., Sadelain, M., 2013). A number of clinical trials have demonstrated that PD-1/PD-L1 antibodies for the treatments of melanoma (Cho et al., 2016; Hamid et al., 2013), multiple myeloma (Badros et al., 2015), leukemia (Pork et al., 2014) have good therapeutic effects (Patent Nos.: 200380109929.8, 201310258289.2, and 201180019629.5). Therefore, modification of the CAR molecule to recognize multiple targets plays an important role in cellular immunotherapy.

### INVENTION DISCLOSURE

It is an object of the present invention to provide a multi-target chimeric antigen receptor (FIG. 1).

The multi-target chimeric antigen receptor provided by the invention consists of a main peptide chain and a co-peptide chain;

The main peptide chain includes an antigen binding domain A, a co-peptide chain domain B, a transmembrane domain C and an intracellular signaling domain D;

The co-peptide chain comprises a main peptide chain linking domain F;

The antigen binding domain A is a polypeptide having a function of binding antigen;

The co-peptide chain linking domain B and the main peptide chain linking domain F are combined with each other;

The co-peptide chain domain B and the main peptide chain domain F are cytokines and corresponding cytokine receptors or cytokines and corresponding fragments of cytokine receptors that can bind to each other;

The transmembrane domain C is a transmembrane region of any membrane-bound protein or a transmembrane region of a transmembrane protein;

The intracellular signaling domain D comprises a primary signaling region.

Among the above multi-target chimeric antigen receptors,

The co-peptide chain further includes an antigen binding domain E;

The antigen binding domain E is a polypeptide having a function to bind an antigen;

The antigen binding domain E and the antigen binding domain A are the same or different.

The intracellular signaling domain D also includes a costimulatory signaling region.

Among the above multi-target chimeric antigen receptors,

The polypeptide having a function to bind to an antigen is an antibody capable of binding an antigen, a ligand capable of binding an antigen, or a receptor capable of binding an antigen.

Among the above multi-target chimeric antigen receptors,

The antibody capable of binding to the antigen is an intact antibody, a Fab of an antibody, an Fc of an antibody, an scFv, a VHH, a full-length polypeptide, or a partial fragment of a VL or VH of an antibody;

The antigen-binding ligand or the antigen-binding receptor is a full-length polypeptide or a partial fragment of the ligand or receptor.

Among the above multi-target chimeric antigen receptors,

The antigens to which the antigen binding domain A and the antigen binding domain E can bind are cell surface antigens or complexes of MHC molecules with polypeptide.

Among the above multi-target chimeric antigen receptors,

The antigen is a cancer associated antigen;

Or in one embodiment, the antigen is brain cancer, bladder cancer, breast cancer, cervical cancer, colorectal cancer, liver cancer, kidney cancer, lymphoma, leukemia, lung cancer, melanoma, metastatic melanoma, mesothelioma , neuroblastoma, ovarian cancer, prostate cancer, pancreatic cancer, renal cancer, skin cancer, thymoma, sarcoma, non-Hodgkin's lymphoma, Hodgkin's lymphoma, uterine cancer-associated antigen, or any combination thereof.

Among the above multi-target chimeric antigen receptors:

The antigen bound by the antigen binding domain A and the antigen binding domain E is as follows: CD123, CD19, CD20, CD22, CD37, ROR1, mesothelin, CD33/IL3Rα, c-Met, BCMA, PSMA, EGFRvIII , GD-2, NY-ESO-1, MAGEA3, (3-human chorionic gonadotropin, AFP, RAGE-1, MN-CA IX, human telomerase reverse transcriptase, RU1, RU2 (AS), hsp70 -2, M-CSF, PSA, PAP, LAGE-1a, p53, Prostein, PSMA, Her2/neu, telomerase, PCTA-1, MAGE, ELF2M, IGF-I, IGF-II, IGF-I receptor , BCR-ABL, E2A-PRL, H4-RET, 1GH-IGK, MYL-RAR, GP100, Mart1, TSP-180, MAGE-4, MAGE-5, MAGE-6, RAGE, p185erbB2, p180erbB-3, c -met, nm-23H1, TAG-72, CA 19-9, CA 72-4, CAM 17.1, NuMa, K-ras, β-catenin, CDK4, Mum-1, p15, p16, 43-9F, 5T4 , 791Tgp72, (3-HCG, BCA225, BTAA, CA 125, CA 15-3\CA 27.29\BCAA, CA 195, CA 242, CA-50, WT1, CD68, FGF-5, G250, EpCAM, MA-50, MG7-Ag, MOV 18, NB/70K, RCAS1, SDCCAG16, TA-90, TAAL6, TAG72, TLP, p53, Ras, TPS, Epstein Barr virus antigen EBVA and human papillomavirus (HPV) E6 and E7 or any combination thereof.

Or the antigen bound by the antigen binding domain A and the antigen binding domain E is a complex of MHC and a short peptide of the above antigen, wherein the antigen is the antigen which the binding domain A and the antigen binding domain E can bind.

Among the above multi-target chimeric antigen receptors,

The antigen bound by the antigen binding domain A and the antigen binding domain E is CD19, CD20, BCMA, CD22, CD33/IL3Ra, Her2, PDL1, NY-ESO-1, GP100, Martl, WT1 or any combination thereof. ;

Or the antigen bound by the antigen binding domain A and the antigen binding domain E is a complex of MHC and the above short peptide of the antigen.

Among the above multi-target chimeric antigen receptors,

The cytokine and corresponding cytokine receptor are cytokines and corresponding cytokine receptors in the gamma chain cytokine family.

Among the above multi-target chimeric antigen receptors,

The cytokine and corresponding cytokine receptor in the yc cytokine family are IL15 and IL15Rα, IL4 and IL4Rα or IL2 and IL2Rα;

Alternatively, the cytokine and the corresponding cytokine receptor in the yc cytokine family are polypeptides in which IL15 and IL15Rα, IL4 and IL4Rα, IL2 or IL2Rα have more than 75% homology.

Among the above multi-target chimeric antigen receptors,

The primary signaling region is the signaling region of CD3-ζ, FcεRIγ, FcRγ, FcRβ, CD3γ, CD36δ, CD3ε, CD5, CD22, CD79a, CD79b, and CD66d, or a combination of above signaling regions;

Or the primary signaling region is a CD3-ζ signaling region with protein sequence of SEQ. ID. NO. 6 or a polypeptide with greater than 75% homology to the CD3ζ signaling region.

Among the above multi-target chimeric antigen receptors,

The costimulatory signaling regions are CD27, CD28, 4-1BB (CD137), OX40, CD30, CD40, PD-1, ICOS, lymphocyte function-associated antigen-1, CD2, CD7, LIGHT, NKG2C, B7-H3 signaling region or the signaling region of the ligand specifically binding to CD83;or any combination of one or more of the above.

Or the costimulatory signaling region is specifically a combination of one or both of a CD28 signaling region and a 4-1BB signaling region;

Or the costimulatory signaling region is a combination of one or both of polypeptides with greater than 75% homology to the CD28 signaling region, the 4-1BB signaling region.

Among the above multi-target chimeric antigen receptors,

In the multi-target chimeric antigen receptor, the antigen binding domain A or E is an Anti-CD19-ScFv, AntiMHC/GP100-VHH, AntiMHC/WT1-VH, AntiCD20-ScFv, AntiCD22-ScFv or PD1 extracellular domain. One or a combination of the two.

The co-peptide linkage domain is IL15Rαsushi, IL4Rα-N-FN3, IL15 or IL4;

The transmembrane domain is a transmembrane region of CD8 or a transmembrane region of CD28;

The intracellular signaling domain is a polypeptide obtained by fusing a CD3ζ signaling region to a 4-1BB signaling region or a CD3ζ signaling region to a CD28 signaling region;

The main peptide chain linking domain is IL15, IL4, IL15Rαsushi or IL4Rα-N-FN3;

Or, in the multi-target chimeric antigen receptor, the antigen binding domain A is antiCD19-ScFv;

The co-peptide chain linking domain is IL15Rαsushi;

The transmembrane domain is a transmembrane region of CD8;

The intracellular signaling domain is a polypeptide obtained by fusing a CD3ζ signaling region with a 4-1BB (CD137) signaling region;

The antigen binding domain E is an extracellular region of PD1;

The main peptide chain joining domain is IL15.

The co-peptide chain domain and the main peptide chain domain are most preferably IL15 and IL15Rαsushi. When IL15 and IL15Rαsushi are used as the co-peptide chain domain (B) and the main peptide chain domain (F), they stimulate the proliferation of NK, cells or T cells, allowing the expression of this multi-targeting chimeric receptor on NK cell or T cell.

The costimulatory signaling region is particularly preferred from the signaling region of one of the following proteins: CD28 and 4-1BB (CD137), or a combination of both.

Among the above multi-target chimeric antigen receptors,

The amino acid sequence of the AntiCD19-ScFv is listed as SEQ. ID. NO. 1;

The amino acid sequence of the AntiMHC/GP100-VHH is listed as SEQ. ID. NO. 15;

The amino acid sequence of the AntiMHC/Mart1-VHH is listed as SEQ. ID. NO. 16;

The amino acid sequence of the AntiCD20-ScFv is listed as SEQ. ID. NO. 17;

The amino acid sequence of the AntiCD22-ScFv is listed as the SEQ. ID. NO. 18;

The amino acid sequence of the AntiMHC/WT1-VH is listed as the SEQ. ID. NO. 19;

The sequence of the extracellular domain of PD1 is listed as SEQ. ID. NO. 2; the amino acid sequence of the IL15Rαsushi is listed as SEQ. ID. NO. 4;

The amino acid sequence of the IL4Rα-N-FN3 is listed as the SEQ. ID. NO. 20;

The amino acid sequence of the transmembrane region of CD8 is listed as SEQ. ID. NO. 5;

The amino acid sequence of the transmembrane region of CD28 is listed as SEQ. ID. NO. 22;

The amino acid sequence of the CD3ζ signaling region is listed as SEQ. ID. NO. 6;

The amino acid sequence of the 4-1BB signaling region is listed as SEQ. ID. NO. 8;

The amino acid sequence of the CD28 signal is listed as SEQ. ID. NO. 7;

The amino acid sequence of IL15 is listed as SEQ. ID. NO. 3;

The amino acid sequence of IL4 is listed as SEQ. ID. NO. 21.

Among the above multi-target chimeric antigen receptors,

The amino acid sequence of the main peptide chain of the multi-target chimeric antigen receptor is any one of SEQ. ID. NO. 9, SEQ. ID. NO. 23, SEQ. ID. NO. 24, SEQ. ID. NO. 25, SEQ. ID. NO. 26, SEQ. ID. NO. 27, SEQ. ID. NO. 28 or SEQ. ID. NO. 29;

The amino acid sequence of the co-peptide chain of the receptor is any one of SEQ. ID. NO. 3, SEQ. ID. NO. 4, SEQ. ID. NO. 10, SEQ. ID. NO. 30, SEQ. ID. NO. 31, SEQ. ID. NO. 32, SEQ. ID. NO. 33 or SEQ. ID. NO. 34.

Another object of the invention is to provide a nucleic acid molecule encoding a multi-target chimeric antigen receptor as described above.

The nucleic acid molecule encoding the multi-target chimeric antigen receptor provided by the present invention comprises a nucleic acid molecule encoding the main peptide chain or a nucleic acid molecule encoding the co-peptide chain.

A nucleic acid sequence encoding a receptor molecule can be obtained using recombinant methods known in the art, such as, for example, by screening a library from a cell expressing the gene, by obtaining the gene from a vector known to include the gene, or by utilizing standard techniques for direct isolation from cells and tissues containing the gene, or synthesis of polynucleotides chemically.

Recombinant vectors, expression cassettes, recombinant bacteria, cells or recombinant viruses containing the above nucleic acid molecules are also within the scope of the present invention.

The above recombinant vector comprises the above nucleic acid sequence or combination. In one embodiment, a nucleic acid encoding a primary peptide chain (X) or a co-peptide chain (Y) can be ligated to a promoter, and the construct is incorporated into an expression vector to express a primary peptide chain (X) or a co-peptide chain (Y). A typical cloning vector comprises a transcriptional and translational terminator, an initial sequence and a promoter that can be used to modulate the expression of a desired nucleic acid sequence. For example, lentiviral vectors are a suitable tool for achieving long-term stable inheritance of genes because they allow long-term, stable integration of genes and their replication in daughter cells. Lentiviral vectors have the added advantage of exceeding vectors derived from oncogenic retroviruses such as murine leukemia viruses because they can transduce non-dividing cells, such as hepatocytes. They also have the added advantage of low immunogenicity. The chimeric antigen receptor provided by the present invention comprises two peptide chains which can be expressed in the same cell in a known manner, including but not limited to co-transfection of respectively vectors encoding a main peptide chain (X) and a co-peptide chain (Y), or an expression vector containing two sets of expression frameworks with a nucleic acid sequence encoding a main peptide chain (X) and a nucleic acid sequence encoding a co-peptide chain (Y), or the nucleic acid sequences encoding a main peptide chain (X) and the co-peptide chain (Y) is ligated in tandem into an expression framework, and both peptide chains are expressed by inserting a ribosome binding site between the nucleic acid sequences of the main peptide chain (X) and the co-peptide chain (Y).

The cell mentioned above, is a prokaryotic cell, a yeast cell or a mammalian cell;

Or the mammalian cell is specifically a human cell;

Or the human cell is specifically an immune cell,

Alternatively, the immune cell is specifically a T cell or an NK cell.

The usage of the above-described multi-target chimeric antigen receptor, the above nucleic acid molecule or the above recombinant vector, expression cassette, recombinant strain, cell or recombinant virus or kit in immunotherapy is also within the scope of protection of the present invention;

Or the usage of the above-described multi-target chimeric antigen receptor, the above nucleic acid molecule or the above recombinant vector, expression cassette, recombinant strain, cell or recombinant virus or kit for preparing an immunotherapeutic product is also the scope of protection of the present invention. .

The usage of the above multi-target chimeric antigen receptor, the above nucleic acid molecule or the above recombinant vector, expression cassette, recombinant strain, cell or recombinant virus or kit for immune cell culture and/or promotion of immune cell proliferation. The scope of invention protection;

The usage of the above-mentioned multi-target chimeric antigen receptor, the above nucleic acid molecule or the above recombinant vector, expression cassette, recombinant strain, cell or recombinant virus or kit in preparing immune cell culture and/or products for promoting immune cell proliferation is also within the scope of the invention.

The use of the above-described multi-target chimeric antigen receptor, the above nucleic acid molecule or the above recombinant vector, expression cassette, recombinant strain, cell or recombinant virus or kit for immunoassay is also within the scope of protection of the present invention;

Or the usage of multi-target chimeric antigen receptor, the nucleic acid molecule or the recombinant vector and the expression cassette, recombinant bacterium, a cell or a recombinant virus or a kit for making an immune detection kit, in the invention is also a protection scope of the present invention;

Or the usage of the multi-target chimeric antigen receptor, the nucleic acid molecule or the recombinant vector and the expression cassette, recombinant bacteria, cells or recombinant viruses or kits for the diagnosis of tumors is also within the protection scope of the invention;

The usage of multi-target chimeric antigen receptor, the nucleic acid molecule or the recombinant vector, the expression cassette and the recombinant bacterium, the recombinant cell or the recombinant virus or the kit in preparation of a product for the treatment or the diagnosis of a tumor is also a protection scope of the invention;

The usage of multi-target chimeric antigen receptor, the nucleic acid molecule or the recombinant vector, the expression cassette and the recombinant bacterium, the recombinant cell or the recombinant virus or the kit in inhibiting or killing tumor cells is also the protection scope of the invention;

The usage of multi-target chimeric antigen receptor, the nucleic acid molecule or the recombinant vector, the expression cassette and the recombinant bacterium, the recombinant cell or the recombinant virus or the kit in preparation of a product for inhibiting or killing tumor cells is also a protection scope of the invention;

The usage of multi-target chimeric antigen receptor, the nucleic acid molecule or the recombinant vector, the expression cassette and the recombinant bacterium, the recombinant cell or the recombinant virus or the kit in inhibiting or killing the target cell expressing the antigen is also the protection scope of the invention;

The usage of multi-target chimeric antigen receptor, the nucleic acid molecule or the recombinant vector, the expression cassette and the recombinant bacterium, the transgenic cell or the recombinant virus or the kit in preparation of a target cell product for inhibiting or killing target cell with the antigen is also a protection scope of the invention.

The immune therapy is used for inhibiting or killing tumor cells through immune cells;

Or, the immune cells are T cells or NK cells and the like;

Or, the antigen is a cancer-related antigen;

Or the antigen is the related antigen of brain cancer, bladder cancer, breast cancer, cervical cancer, colorectal cancer, liver cancer and kidney cancer, lymphoma, leukemia, lung cancer, melanoma, metastatic melanoma, mesothelioma and neuroblastoma, ovarian cancer, prostate cancer, pancreatic cancer, kidney cancer, skin cancer, thymoma and sarcoma, a non-Hodgkin's lymphoma, a Hodgkin's lymphoma, an uterine cancer or any combination thereof;

Or, the tumor is any one of the following: brain cancer, bladder cancer, breast cancer, cervical cancer, colorectal cancer, liver cancer and kidney cancer, lymphoma, leukemia, lung cancer, melanoma, metastatic melanoma, mesothelioma and neuroblastoma, ovarian cancer, prostate cancer, pancreatic cancer, kidney cancer, skin cancer, thymoma and sarcoma, the non-Hodgkin's lymphoma, the Hodgkin's lymphoma and the uterine cancer; or the combination of above.

Or, the target cells are prokaryotic cells, yeast cells or mammalian cells;

Or, the mammal cells are specifically human cells;

Or, the human cells are immune cells,

Or, the immune cells are specifically T cells or NK cells

The antigen in the target cell expressing the antigen is an antigen which can be bound by a multi-target chimeric antigen receptor of IL15 receptor, IL15Rαsushi for short, and has the ability to bind to the IL15.

In the invention, the IL4Rα-N-F3 is a fragment from the alpha chain of IL4 receptor, with the function of binding to the IL4.

The antigen binding domain (A) of the main peptide chain and the co-peptide chain linking domain (E) of the present invention have a function to bind to an antigen. The antigen bound by antigen-binding domain is a protein produced by tumor cells causing immune responses, in particular T-cell-mediated immune responses. The selection of the antigen binding domain of the invention will depend on the specific type of the disease to be treated. The tumor antigen is well known in the field:

In one embodiment, the tumor antigens mentioned herein include, for example, neuroglioma-related antigens, cancer embryo antigen (CEA)), beta-human chorionic gonadotropin and alpha-fetal protein (AFP)), lectin-reacted AFP, thyroglobulin, RAGE-1, MN-CA/IX, human telomerase reverse transcriptase, RU1, RU2 (AS), enterocarboxylesterase, mut HSP70-2, M-CSF, prostate enzyme, prostate-specific antigen (PSA), PAP, NY-ESO -1, LAGE-la, p53, prostein, PSM, Her2/neu, Survivin, telomerase, prostate-cancer tumor antigen -1 (PCTA -1),MAGE, ELF2M, neutral leukocyte elastase, Ephrin B2, CD22, an insulin growth factor (IGF))-I, IGF-II, IGF-I receptors and Mesothelin.

In one embodiment, the tumor antigen includes one or more antigen cancer epitopes associated with a malignant tumor. A malignant tumor expresses many proteins that can be used as a target antigen for immune attack. These molecules include, but are not limited to, tissue-specific antigen such as MART -1, tyrosinase and gp100 in melanoma, and prostate acid phosphatase (PAP) and prostate-specific antigen (PSA)in prostate cancer). Other target molecules belong to the group of transformation-related molecules, such as oncogenes HER −2/Neu ERB −2. The target antigen of the other group is a fetal cancer antigen such as carcinoembryonic antigen (CEA). InB-cell lymphoma, and the tumor-specific individual genotype immunoglobulin forms a true tumor-specific immunoglobulin antigen which is unique to an individual tumor. The B-cell differentiation antigens such as CD19, CD20 and CD37 are other candidates of target antigens in B-cell lymphoma. Some of these antigens (CEA, HER -2, CD19, CD20, individual genotypes) have been successfully used as targets for a passive therapy using a monoclonal antibody.

In one embodiment, the tumor antigen mentioned in the invention can also be a tumor-specific antigen (TSA) or a tumor-associated antigen (TAA). The TSA is unique to tumor cells and does not occur on other cells of the body. By contrast, the TAA-related antigens are not unique to tumor cells and can also be expressed on normal cells under the condition that the immune tolerance state of the antigen cannot be induced. The antigen expression on the tumor can occur under the disease condition that the immune system can respond to the antigen. TAA can be the antigen appeared during embryonic development and expressed on normal cells when the immune system is immature and cannot respond, or they can be antigens that are normally present at extremely low levels on normal cells while expressed at higher levels on tumor cells;

Examples of TSA or TAA antigens include but not limited tothe following: differentiation antigens such as MART-1/MelanA, gp100 (Pmel 17), tyrosinase, TRP-1, TRP-2,tumor-specific multi-pedigree antigens such as MAGE-1, MAGE-3, BAGE, GAGE-1, GAGE-2, p15;over-expressed embryonic antigens such as CEA, over-expressed cancerogenic genes and mutated tumor-suppressor genes such as p53, Ras, HER-2/neu; unique tumor antigens generated by chromosome translocation such as BCR-ABL, E2A-PRL, H4-RET, IGH-IGK, MYL-RAR; And viral antigens, such as Epstein Barr virus antigen EBVA and human papilloma virus (HPV) antigen E6 and E7. Other protein antigens including TSP-180, MAGE-4, MAGE-5, MAGE-6, RAGE, NY-ESO-1, p185erbB2, p180erbB-3, c-met, nm-23H1, PSA, TAG-72, CA 19-9, CA 72-4, CAM 17.1, NuMa, K-ras, beta-catenin, CDK4, Mum-1, p15, p16, 43-9F,5T4, 791Tgp72, alpha-fetal protein, beta-HCG,BCA225, BTAA, CA 125, CA 15-3/CA, 27.29/BCAA, CA 195, CA 242, CA-50, CAM43, CD68/P1, CO-029, FGF-5, G250, Ga733/EpCAM, HTgp -175, M344, MA-50, MG7-Ag,MOV18, NB/70K, NY-CO-1, RCAS1, SDCCAG16, TA-90,TA-90 \ Mac -2 binding protein\ ring-philic protein C-related protein, TAAL6, TAG72and TPS.

In one embodiment, the tumor antigen mentioned in the present invention can be a compound of MHC with a said antigen peptide fragment, including but is not limited to, HLA-gp100 complex, HLA-MART-1 complex and HLA-WT1 complex.

In one embodiment, antigen binding domain in said main peptide chain (A) or an antigen binding domain in the co-peptide chain (E) is an antibody which can be combined with an antigen, a ligand, a receptor, a polypeptide with an antigen binding capability or any combination thereof.

Antibody can be complete peptide chain or peptide fragment of Ig, Fab and scfv or any combination thereof. The ligand or the receptor can be a complete peptide chain, a peptide fragment or any combination thereof.

The co-peptide chain connecting domain (B) and the main peptide chain connecting domain (F) is a pair of peptide fragments with mutual binding functions. The peptide fragment with the mutual binding function can be a pair of receptor and ligand which can be combined with each other or a pair of antibody and the antigen which can be combined with each other. The mutually-combined receptor and ligand include, but are not limited to, IL15 and IL15R alpha, IL4 and IL4R, IL2 and IL2Ralpha, CD16 and IgGFc, CD32 and IgGFc, CD64 and IgGFc.

Wherein the transmembrane domain (C) can be derived from any membrane-binding protein or the transmembrane region of a transmembrane protein. For a transmembrane domain, in some instances, a transmembrane domain may be selected, or modification is carried out through amino acid replacement, so that the structure domain can be prevented from being bound to the same or different transmembrane region of a surface expressed membrane protein in order to reduce the steric hindrance. The transmembrane domain can be derived from a natural source or a synthetic source. In a natural source, the domain can be derived from any membrane-binding protein or transmembrane protein. The transmembrane region specifically used in the present invention may originate from that including but not limited to, alpha, beta or zeta chain of T cell receptor, CD28, CD3epsilon, CD45, CD4, CDS, CD8, CD9, CD16, CD22, CD33, CD37, CD64, CD80, CD86, CD134, CD137, CD154 and ICOS.

Wherein the intracellular signal transduction domain (D) comprises a signal transduction region, and may also comprise a co-stimulation signal transduction region or a combination thereof. The intracellular signal transduction domain refers to a protein fragment which transduces effector function signal and guides a cell to implement a specific function. For example, effector functions of T cells can be cell dissolution activity or auxiliary activity through cytokine secretion. The whole intracellular signaling structure domain can be used, but in many instances, the whole chain does not need to be used. A truncated version of the intracellular signal transduction domain can be used to replace a whole chain, as long as it has the function signal of a transduction effector.

Primary cell plasma signal transduction sequence to stimulate primary activation of immune cells in a stimulating manner or in an inhibition manner. A primary cell signal transduction sequence acting in a stimulation manner may comprise a signal transduction motif, which is known as an activation motif based on immune receptor tyrosine or an ITAM.

Examples of ITAM with primary cytoplasmic signal conduction in the present invention include TCR, FccRIy, FcRy, Fen, CD3y, CD36, CD3c, CD5, CD22, CD79a, CD79b, CD66d or their combination. Preferably, the cytoplasmic signal transduction molecule of the CAR construct disclosed by the invention is derived from CD3;

Co-stimulating signal transduction region in the invention refers to intracellular domains comprising co-stimulating molecules. The co-stimulating molecule is a cell surface molecule required by effective response of a lymphocyte to an antigen, and not an antigen receptor or a ligand thereof. Examples of such molecules include CD27, CD28, and 4-1 BB (cd137), OX40, CD30, CD40, PD -1, ICOS, lymphocyte function related antigen-1 (LFA -1), CD2, CD7, LIGHT, NKG2C, B7-H3 and a ligand specifically bound to CD83, etc. and the like. Thus, although the present invention mainly uses 4-1 BB and CD28 as co-stimulating signal molecules, but other co-stimulating elements are also within the scope of the present invention.

The cytoplasmic signaling sequences of cell signal transmission part of the multi-target chimeric antigen receptor can be connected with each other in random or in a specified sequence.

In one embodiment, the intracellular signal transduction domain comprises a cd3-zeta signal transduction domain. In another embodiment, an intracellular signal transduction region includes cd3-zeta, and the signal fragment of 4-1 BB. In another embodiment, wherein the intracellular signal transduction structure comprises a signal part of CD3-zeta and the signal transduction structure of CD28.

Short oligopeptides or polypeptides can be used as connecting peptides between modules in a multi-target chimeric antigen receptor, so that the function of the multi-target chimeric antigen receptor cannot be influenced, and preferably, the length of the connecting peptide is between 2 and 10 amino acids, wherein the amino acids of the connecting peptide are preferably glycine and serine.

### BRIEF DESCRIPTION OF THE DRAWINGS

FIG. 1 The schematic diagram of multi-target chimeric antigen receptor. The multi-target chimeric antigen receptor consists of a main peptide chain and an co-peptide chain, wherein the main peptide chain comprises an antigen binding domain A, aco-peptide chain connecting domain B, a transmembrane domain C and an intracellular signal transduction domain D; and the co-peptide chain comprises a main peptide chain connecting domain F, which can also comprise an antigen binding domain E.

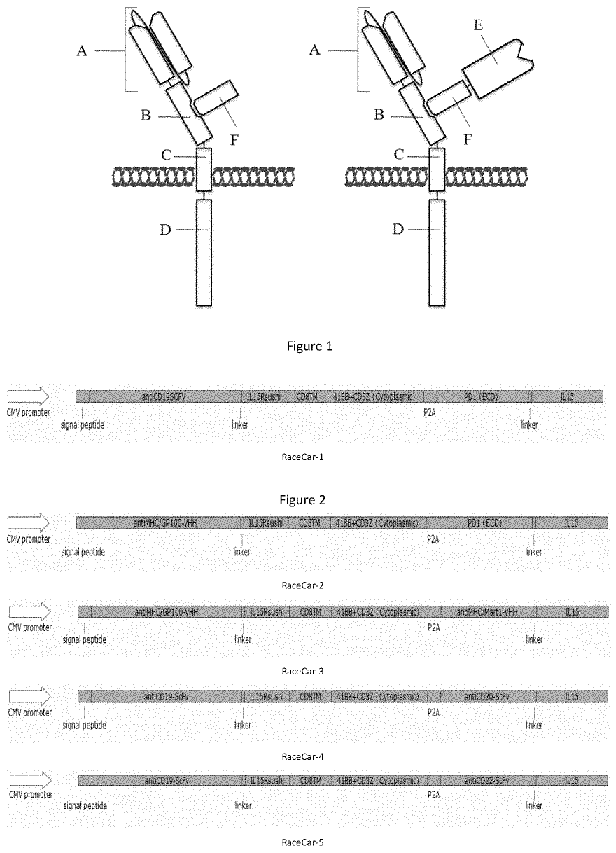

FIG. 2 Schematic diagram of RaceCar-1 expression cassette. The invention discloses a RaceCar-1 expression cassette which is composed of a promoter and an encoding gene of RaceCar-1.

FIG. 3 Schematic diagram of different RaceCar expression cassette. The other RaceCar expression cassette is composed of a promoter and an encoding gene of RaceCar.

FIG. 4 FACS analysis to verify RaceCar expressed on cell surface. The horizontal coordinate in the figure represents the IL level, the T cell and the NK92 are not infected and used as negative control and there is no expression of IL15 on the surface detected. The expression of different levels of IL15 can be detected on the surfaces of T cells and NK92 cells infected with RaceCar virus. Since IL15 is the domain molecule in the co-peptide chain of RaceCar (except RaceCar-8, RaceCar-12 and RaceCar-13), IL -15 can be only detected on the surface of the cell when it binds to the domain of the main peptide chain expressed on the surface of the cell, which demonstrated that RaceCar can be expressed on the surface of T cells and NK92 cells, and expressed IL15 has immunological activity. Meanwhile, IL15 is on a main peptide chain of RaceCar -12 and RaceCar-13, and can be directly detected.

FIG. 5 RaceCar expression demonstrated by Western blot. Samples of RaceCar-1-293T, RaceCar-2-293T, RaceCar-6-293T, RaceCar-7-293T, and RaceCar-10-293T, were loaded on lanel-5 respectively. The sixth lane is 293T cells without transfection used as a negative control. The lower strip is a beta-actin reference. A specific strip of PD1can be seen in the lane 1 to the lane 5, which indicates that the RaceCars are expressed on transfected cells.

FIG. 6 Demonstrated killing capability of RaceCar-NK92 cells. The horizontal coordinate of the graph is the ratio of effector cells to target cells, and the vertical coordinate is the killing efficiency. The RaceCar-1-NK92, RaceCar-2-NK92, RaceCar-3-NK92, RaceCar-4-NK92 and RaceCar-5-NK92 all could can kill 3m-CD19-luc cells and killing efficiency is higher than that of NK92, which demonstrates that the multi-target chimeric antigen receptor RaceCar-1 targeting to CD19 and PD-L1 is expressed in the NK92 cells, and has a biological function. There is reduced killing ability of experimental group with adding anti-CD19 antibody onRaceCar-1-nk92 while the killing capacity of the RaceCar-2-NK92 is unchanged since anti-CD19 antibody blocks the binding of RaceCar-1 to the target cell, so that the killing capability of the racemase-l-NK92 on the target cell is inhibited, which proves that the RaceCar-NK92 has specificity on killing 3m-CD19-luc cells.

FIG. 7 The comparison of killing efficiency against target cells between RaceCar-T and Car-T cells. The horizontal coordinate of the graph is the ratio of effector cells to target cells, and the vertical coordinate is the killing efficiency. RaceCar-1-T, RaceCar-4-T, RaceCar-5-T cells can kill k562-cd19-luc cells, which demonstrates that RaceCar-1, RaceCar-4 andRaceCar-5are expressed on T cells and have a biological function; and the killing capacity of the RaceCar-T is higher than that of common Car-T cells;

FIG. 8 Functional verification of different forms of RaceCar-T and RaceCar-NK92 targeting to WIC/WT-1 complex. The horizontal coordinate of the graph is effector cell types, and the vertical coordinate is the killing efficiency. Both RaceCar-NK92 and RaceCar-T cells can kill BV173-luc cells, and killing efficiency is higher than that of non-infected T cells or NK92 cells, demonstrating that RaceCar-6, RaceCar-7, RaceCar-8, RaceCar-9, RaceCar-10, RaceCar-11, RaceCar-12, RaceCar-13 and RaceCar-14 can express on both T cells and NK92 cells, and have biological function to direct the killing for corresponding target cells;

FIG. 9 FACS analysis for the ability of RaceCar to induce the proliferation of immune cells. On day zero the fluorescence signals of the four groups of cells are almost the same. T cell group is a negative control group, and its fluorescence intensity on the fifth day is basically unchanged compared with that on day zero. The FACS analysis of T cell +IL-2 group as a positive control on the fifth day can be seen in the figure, the fluorescence signal is weakened comparing to that on day 0 and the cells show different fluorescence staining, which demonstrates that cells are shown the phenotypes of division and proliferation. The fluorescence signal distribution of RaceCar-1-T and RaceCar-2-T is similar to that of the positive control group, indicating thatRaceCar-1-T and RaceCar-2-T can proliferate in a culture medium without IL2 and RaceCar has ability to promote the proliferation of immune cells such as T cells and the like.

### THE BEST WAY FOR THE APPLICATION OF THE INVENTION

The experimental methods used in the following examples are conventional methods if not specifically described.

The materials, reagents and the like used in the embodiments of the present invention can be obtained from commercial resources, if there is no specific description.

### EXAMPLE 1

### The Structure of Multi-Target Chimeric Antigen Receptor and Expression Vector Construction

The multi-target chimeric antigen receptor (RaceCar) protein is obtained by complexing a main peptide chain X and a co-peptide chain Y;

Main peptide chain (X) comprises antigen binding domain (A), co-peptide chain connecting domain (B) and transmembrane domain (C) and an intracellular signal transduction domain (D), and the co-peptide chain(Y) comprises an antigen binding domain (E) and a main peptide chain connecting domain (F). The co-peptide chain connecting domain (B) and a main peptide chain connecting domain (F) complementary bind to each other, enabling that the main peptide chain X and the co-peptide chain Y to polymerize to form a multi-target chimeric antigen receptor (RaceCar).

Multi-target chimeric antigen receptor (RaceCar-1) targeting to both CD19 and PD-Ll positive cells is obtained by polymerizing a main peptide chain X1 and a co-peptide chain Y1;

In RaceCar-1, scfv of anti-CD19 (SEQ. ID. NO. 1) is chosen as antigen binding domain (Al) of main peptide chain X1, and co-peptide chain connecting domain (B) adopts the sushi fragment (SEQ. ID. NO. 4) of IL15R [alpha],the transmembrane of CD8 (SEQ. ID. NO. 5) is chosen as the transmembrane domain (C) and an intracellular signal transduction domain (D) is constructed by linking a 41-BB signal transduction domain (SEQ. ID. NO. 8) to a CD3 zeta signaling region (SEQ. ID. NO. 6). The antigen binding domain (E) of co-peptide chain Y1 adopts an extracellular region (SEQ. ID. NO. 2) of PD1, a receptor of the PDL 1. IL15 (SEQ. ID. NO. 3) is used as the main peptide chain connecting domain (F). Finally, a main peptide chain X1 (SEQ. ID. NO. 9) connecting with the co-peptide chain Y1 (SEQ. ID. NO. 10) is made.

2.) A secretory signal peptide (SEQ. ID. NO. 11) is inserted before N-terminal of the main peptide chain X1 and it was artificially synthesizing as the coding nucleic acid DNA-X1 (SEQ. ID. NO. 13). Similarly, a 2A signal peptide (SEQ. ID. NO. 12) is added in front of N-terminal of co-peptide chain Yl, wherein the coding sequence is synthesized as sequence DNA-X2 (SEQ. ID. NO. 14);

3.) BamHI and Hindlll sites were added to each end of DNA-X1 and cut by BamHI and Hindlll to obtain the fragment of the DNA-X1; Similarly, a Hindlll site is added to the 5′-end of DNA-X2 sequence, and an EcoRI site is added at the 3′-end, and cut by Hindlll and EcoRI to obtain a nucleic acid fragment of the DNA-X2;

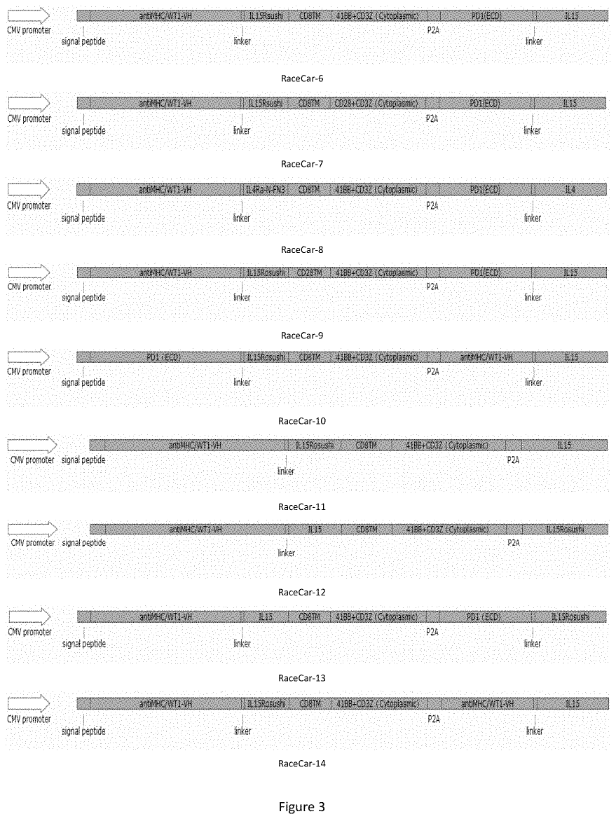

4.) The two nucleic acid fragments obtained in 3 are inserted into the vector pFUGW (Addgene, USA) cut by the same combination of enzymes, the ligated product is transformed into escherichia coli, individual colonies are picked and screened using PCR (polymerase chain reaction) analysis and finally a recombinant vector pFUGW-RaceCar-1 is obtained. The recombinant vector pFUGW-RaceCar-1 replaces the nucleic acids for expressing RaceCar-1 (the coding nucleic acid is composed of a SEQ. ID. NO. 13, an AAGCTT and a SEQ. ID. NO. 14, and the last nucleotide of the SEQ. ID. NO. 13 is adjacent to the first base group of the AAGCTT, the first nucleotide of the SEQ. ID. NO. 14 is adjacent to the last base of the AAGCTT that is a digestion site for HindIII) for a fragment between BamHI and EcoRI cleavage sites of pFUGW vector. RaceCar expression cassette is formed by coding nucleic acids of RaceCar-1 and the promoter in pFUGW(FIG. 2)

5.) Designing other multi-target chimeric antigen receptors in the same manner as shown in Table 1:

TABLE 1
The structure of multi-targeted chimeric antigen receptor (RaceCar) structure

| | Domain | | | | | |
|---|---|---|---|---|---|---|
| | | Co-peptide | | intracellular | | |
| | Antigen | chain | | signaling | Antigen- | Main peptide |
| | binding | connecting | Transmembrane | structure | binding | chain-linking |
| Name | domain A | domain B | domain C | domain D | domain E | domain F |
| RaceCar-1 | AntiCD19- | IL15Rαsushi | Transmembrane | 41BB + | extracellular | IL15 |
| | ScFv | | domain of CD8 | CD 3ζ | region | |
| | | | | | of PD1 | |
| RaceCar-2 | AntiMHC/G | IL15Rαsushi | Transmembrane | 41BB + | extracellular | IL15 |
| | P100-VHH | | domain of CD8 | CD 3ζ | region | |
| | | | | | of PD1 | |
| RaceCar-3 | AntiMHC/G | IL15Rαsushi | Transmembrane | 41BB + | AntiMH | IL15 |
| | P100-VHH | | domain of CD8 | CD 3ζ | C/Mart1- | |
| | | | | | VHH | |
| RaceCar-4 | AntiCD19- | IL15Rαsushi | Transmembrane | 41BB + | AntiCD2 | IL15 |
| | ScFv | | domain of CD8 | CD 3ζ | 0-ScFv | |
| RaceCar-5 | AntiCD19- | IL15Rαsushi | Transmembrane | 41BB + | AntiCD2 | IL15 |
| | ScFv | | domain of CD8 | CD 3ζ | 2-ScFv | |
| RaceCar-6 | AntiMHC/W | IL15Rαsushi | Transmembrane | 41BB + | extracellular | IL15 |
| | T1-VH | | domain of CD8 | CD 3ζ | region | |
| | | | | | of PD1 | |
| RaceCar-7 | AntiMHC/W | IL15Rαsushi | Transmembrane | CD28 + | extracellular | IL15 |
| | T1-VH | | domain of CD8 | CD 3ζ | region | |
| | | | | | of PD1 | |
| RaceCar-8 | AntiMHC/W | IL4Rα-N-FN3 | Transmembrane | 41BB + | extracellular | IL4 |
| | T1-VH | | domain of CD8 | CD 3ζ | region | |
| | | | | | of PD1 | |
| RaceCar-9 | AntiMHC/W | IL15Rαsushi | Transmembrane | 41BB + | extracellular | IL15 |
| | T1-VH | | domain of CD8 | CD 3ζ | region | |
| | | | | | of PD1 | |
| RaceCar-10 | extracellular | IL15Rαsushi | Transmembrane | 41BB + | AntiMH | IL15 |
| | region of | | domain of CD8 | CD 3ζ | C/WT1-VH | |
| | PD1 | | | | | |
| RaceCar-11 | AntiMHC/W | IL15Rαsushi | Transmembrane | 41BB + | — | IL15 |
| | T1-VH | | domain of CD8 | CD 3ζ | | |
| RaceCar-12 | AntiMHC/W | IL15 | Transmembrane | 41BB + | — | IL15Rαsushi |
| | T1-VH | | domain of CD8 | CD 3ζ | | |
| RaceCar-13 | AntiMHC/W | IL15 | Transmembrane | 41BB + | extracellular | IL15Rαsushi |
| | T1-VH | | domain of CD8 | CD 3ζ | region | |
| | | | | | of PD1 | |
| RaceCar-14 | AntiMHC/W | IL15Rαsushi | Transmembrane | 41BB + | AntiMH | IL15 |
| | T1-VH | | domain of CD8 | CD 3ζ | C/WT1-VH | |

On Table 1, the amino acid sequence of AntiMHC/GP100-VHH is listed on SEQ. ID. NO. 15, and the amino acid sequence of AntiMHC/Mart-1-VH is listed in SEQ. ID. NO. 16, the amino acid sequence of the AntiCD20-scfv is listed on SEQ. ID. NO. 17, and the amino acid sequence of the AntiCD22-scfv is listed on SEQ. ID. NO. 18, the amino acid sequence of the AntiMHC/WT1-VH is listed on SEQ. ID. NO. 19, and the sequence of the IL4Ralpha-N-FN3 amino acid sequence is as shown in the SEQ. ID. NO. 20, the sequence of IL4 is listed on SEQ. ID. NO. 21, and the sequence of CD28 transmembrane region is listed as SEQ. ID. NO. 22.

The sequences of different main peptide chain X are listed as follows: main peptide chain X2 (the amino acid sequence is SEQ. ID. NO. 23); the main peptide chain X3 (the amino acid sequence is SEQ. ID. NO. 24); X4 (the amino acid sequence is SEQ. ID. NO. 25); X5 (the amino acid sequence is SEQ. ID. NO. 26): X6 (the amino acid sequence is a SEQ. ID. NO. 27); X7 (the amino acid sequence is SEQ. ID. NO. 28), X8 (the amino acid sequence is a SEQ. ID. NO. 29); and the co-peptide chain Y2 (the amino acid sequence is a SEQ. ID. NO. 30), Y3 (the amino acid sequence is SEQ. ID. NO. 31); Y4 (the amino acid sequence is SEQ. ID. NO. 32), Y5 (the amino acid sequence is SEQ. ID. NO. 33); Y6 (the amino acid sequence is the sequence IL15 and listed on SEQ. ID. NO. 3); Y7 (IL15Ralpha-sushi, the amino acid sequence is SEQ. ID. NO. 4); Y8 (the amino acid sequence is SEQ. ID. NO. 34), and Y9 (the amino acid sequence is SEQ. ID. NO. 39).

Wherein:

RaceCar-2 is composed of a main peptide chain X2 and a co-peptide chain Y1;

RaceCar-3 is composed of a main peptide chain X2 and a co-peptide chain Y2;

RaceCar-4 is composed of a main peptide chain X1 and a co-peptide chain Y3;

RaceCar-5 is composed of a main peptide chain X1 and a co-peptide chain Y4;

RaceCar-6 is composed of a main peptide chain X3 and a co-peptide chain Y1;

RaceCar-7 is composed of a main peptide chain X4 and a co-peptide chain Y1;

RaceCar-8 is formed by main peptide chain X5 and a co-peptide chain

Y9;

RaceCar-9 is composed of a main peptide chain X6 and aco-peptide chain Y1;

RaceCar-10 is composed of a main peptide chain X7 and a co-peptide chain Y5;

RaceCar-11 is composed of a main peptide chain X3 and a co-peptide chain Y6;

RaceCar-12 is composed of a main peptide chain X8 and aco-peptide chain Y7;

RaceCar-13 is composed of a main peptide chain X8 and aco-peptide chain Y8;

RaceCar-14 is composed of a main peptide chain X3 and a co-peptide chain Y5.

The same as RaceCar-1, when the expression vectors for other RaceCars are constructed, a secretory leader sequence (SEQ. ID. NO. 11) needs to be added before N-terminal sequence of the main peptide chain of other RaceCars wherein a 2A signal peptide is added in front of the amino acid sequence of the co-peptide chain; The specific coding nucleic acid sequences are as follows:

The nucleic acid sequence expressing RaceCar-2 is composed of DNA-X2 (SEQ. ID. NO. 40), AAGCTT and DNA-Y1 (SEQ. ID. NO. 14);

The nucleic acid sequence for expressing RaceCar-3 is composed of DNA-X2 (SEQ. ID. NO. 40), AAGCTT and DNA-Y2 (SEQ. ID. NO. 47);

The nucleic acid sequence expressing RaceCar-4 is composed of DNA-X1 SEQ. ID. NO. 13), AAGCTT and DNA-Y3 (SEQ. ID. NO. 48);

The nucleic acid sequence expressing RaceCar-5 is composed of DNA-X1 (SEQ. ID. NO. 13), AAGCTT and a DNA-Y4 (SEQ. ID. NO. 49);

The nucleic acid sequence expressing RaceCar-6 is composed of DNA-X3 (SEQ. ID. NO. 41), AAGCTT and DNA-Y1 (SEQ. ID. NO. 14);

The nucleic acid sequence for expressing RaceCar-7consists of DNA-X4 (SEQ. ID. NO. 42), AAGCTT and DNA-Y1 (SEQ. ID. NO. 14);

The nucleic acid sequence for expressing RaceCar-8 consists ofDNA-X5 (SEQ. ID. NO. 43), AAGCTT and DNA-Y9 (SEQ. ID. NO. 50);

The nucleic acid sequence expressing RaceCar-9 is composed of DNA-X6 (SEQ. ID. NO. 44), AAGCTT and DNA-Y1 (SEQ. ID. NO. 14);

The nucleic acid sequence for expressing RaceCar-10 is composed of DNA-X7 (SEQ. ID. NO. 45), AAGCTT and DNA-Y5 (SEQ. ID. NO. 51);

The nucleic acid sequence for expressing RaceCar-1 lis composed of DNA-X3 (SEQ. ID. NO. 41), AAGCTT and DNA-Y6 (SEQ. ID. NO. 52);

The nucleic acid sequence for expressing RaceCar-12is composed of DNA-X8 (SEQ. ID. NO. 46), AAGCTT and a DNA-Y7 (SEQ. ID. NO. 53);

The nucleic acid sequence for expressing RaceCarl3is composed of DNA-X8 (SEQ. ID. NO. 46), AAGCTT and DNA-Y8 (SEQ. ID. NO. 54);

The nucleic acid sequence for expressing RaceCarl4is composed of DNA-X3 (nucleic acid SEQ. ID. NO. 41), AAGCTT and DNA-Y5 (SEQ. ID. NO. 51);

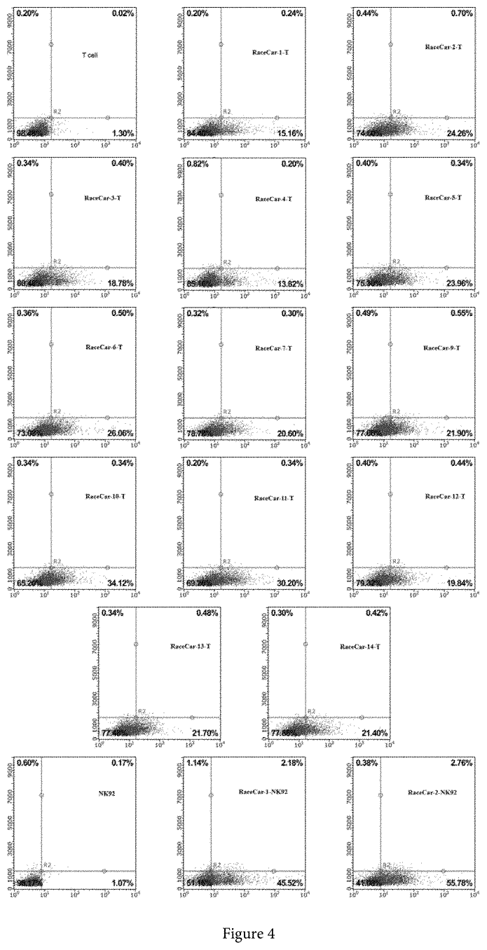

The nucleic acid sequences for expressing other multi-targeted chimeric antigen receptors are obtained in the same manner; and recombinant vectors ranging from pFUGW-RaceCar-2 to pFUGW-RaceCar-14 are constructed. In recombinant vectors there are expression cassettes for other individual multi-target chimeric antigen receptor RaceCar (FIG. 3).

Recombinant vectors ranging from pFUGW-RaceCar-2 to pFUGW-RaceCar-14 come from replacing a fragment between BamHI and EcoRI cleavage sites of pFUGW vector with nucleic acid sequences for expressing RaceCar2 to RaceCar14 respectively.

### EXAMPLE 2

### FACS Analysis to Confirm RaceCar Expression on Cells

1.) RaceCar-1-NK92, RaceCar-2-NK92, RaceCar-3-NK92, RaceCar-4-NK92, RaceCar-5-NK92, RaceCar-6-NK92, RaceCar-7-NK92, RaceCar-9-NK92, RaceCar-10-NK92, RaceCar-11-NK92, RaceCar-12-NK92, RaceCar-13-NK92, RaceCar-14-NK92, RaceCar-1-T, RaceCar-2-T, RaceCar-3-T, RaceCar-4-T, RaceCar-5-T, RaceCar-6-T, RaceCar-7-T, RaceCar-9-T, RaceCar-10-T, RaceCar-11-T, RaceCar-12-T, RaceCar-13-T, RaceCar-14-T cells obtained in example 4, are suspended in PBS (phosphate buffered saline) respectively, the concentration of cells is controlled to be in the range of 1*10\hat{ }6/ml.

2.) 1.5 μl of anti-hIL-15 PE conjugates is (R&D, IC2471P) is added into 200 μl of treated cells and incubates on ice for 30 minutes. The supernatants are removed by centrifugation and the cells are re-suspended by adding an equal amount of PBS (phosphate buffer saline).

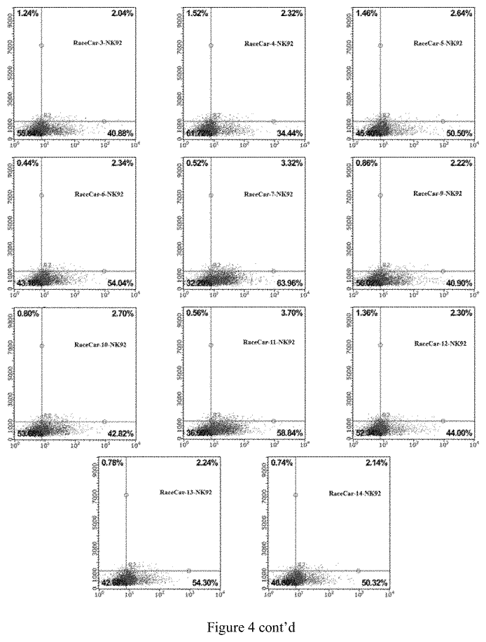

3.) FACS analysis to verify RaceCar expressed on cell surface (FIG. 4). The horizontal coordinate in the figure represents the IL15 level, the T cell and the NK92 are not infected and used as negative control and there is no expression of IL15 on the surface detected. The expression of different levels of IL15 can be detected on the surfaces of T cells and NK92 cells infected with RaceCar virus. Since IL15 is the domain molecule in the co-peptide chain of RaceCar (except RaceCar-8, RaceCar-12 and RaceCar-13), IL-15 can be only detected on the surface of the cell when it binds to the domain of the main peptide chain expressed on the surface of the cell, which demonstrated that RaceCar can be expressed on the surface T cells and NK92 cells, and expressed IL15 has immunological activity. Meanwhile, IL15 is on a main peptide chain of RaceCar -12 and RaceCar-13, and can be directly detected.

### EXAMPLE 3

### The Expression of RaceCar Demonstrated by Western Blot

1.) 1.2*10\hat{ }6 of Lenti-X-293T cells (Clonetech, 632180, hereinafter referred to as 293T for short) are laid in a 6-wellplate, and cultured overnight at the temperature of 37° C. and 5% of CO₂;

2.) 293T cells described as above 1 are transfected respectively with vectorspFUGW-RaceCar-1, pFUGW-RaceCar-2,pFUGW-RaceCar-6, pFUGW-RaceCar-7 and pFUGW-RaceCar-10 mentioned in example 1 using lipofectamine 3000, and are named as RaceCar-1-293T, RaceCar-2-293 T, RaceCar-6-293T, RaceCar-7-293Tand RaceCar-10-293T and cultured with 5% of CO₂ for 48 hours at the temperature of 37° C.

3.) 5×10\hat{ }6 of RaceCar-1-293T, RaceCar-2-293T, RaceCar-6-293T, RaceCar-7 -293T and RaceCar-10-293Twere taken and centrifuged for 10 minutes at 1500 R, and the supernatants were discarded.

4.) 200 μl of cell lysis solution (Beyotime, Shanghai, P0013B) was added onto each cells and incubated on ice for 30 minutes. The cells were centrifuged at 8000 g for 5 minutes. 20 μl of supernatants for each samples was mixed with 5× protein electrophoresis buffer and incubated for 5 minutes at 95° C. to be used as electrophoresis samples.

5.) SDS-PAGE electrophoresis was carried out and gel was transferred onto membrane at 100V, 200 mA for one hour.

6.) Protein transferred PVDF (polyvinylidene fluoride) membrane was blocked in 5% skimmed milk of PBS overnight. The membrane was washed 3 times, each time for 15 minutes with PBST on a shaker.

7.) PVDF with transferred proteins was incubated with 0.1% anti-PD1 antibody (R&D MA1086-100)dissolved in 1% skimmed milk of PBS solution for 1 hour at the shaking condition. The membrane was washed with PBST for three times each for 15 minutes.

8.) Washed membrane was incubated with 0.1% HRP conjugated anti-mouse IgG(H+L) antibody (Bejotime, Shanghai, A0216) dissolved in 1% skimmed milk of PBS solution for 1 hour at the shaking condition. The membrane was washed with PBST for three times each for 15 minutes.

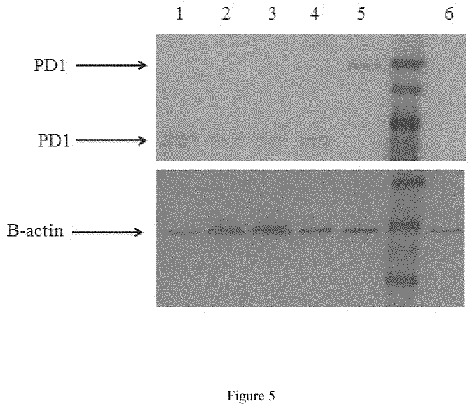

9.) The membrane was visualized with a W-TMB kit (Sangon Biotech, Shanghai, C510025-0005) and the result is shown in FIG. 5. The first to fifth lane samples belong to respectively RaceCar-1-293T, RaceCar-2-293T, RaceCar-6-293T, RaceCar-7-293T and RaceCar-10-293T and a negative control, 293T cells without transfection is located on lane 6. The lower stained line is a beta-actin reference. As shown on FIG. 5, a specific line as PD1 staining can be found in the lane 1 to the lane 5, demonstrating that RaceCar was well expressed on the surface of transfected cells.

### EXAMPLE 4

### Packaging of Lentivirus with pFUGW-RaceCar and Cell Infection

1.) Taking RaceCar-1 as an example, 293T cells were cultured overnight to the density of 70-80% for transfection. pCMV-VSV-g, pCMV-deltaR8.91 (pCMV-VSV-G and pCMV-delta R8.91 were Addgene products)providing virus shell protein were mixed withpFUGW-RaceCar-1 prepared from example 1 according to the ratio of pCMV-VSV-g: pCMV-deltaR8.91: pFUGW-RaceCar-1=1:3:4 to obtain 40 ug of co-transfection plasmids.

2.) In a 15 ml of centrifugal tube marked as Tubel, co-transfection plasmids described as above 1 were added and serum-free DMEM were added up to 1.5 ml, and in an another 15 ml of centrifugal tube marked as Tube2,120 μl of PEI solution (Sigma, GF70215828,1mg/ml was added and serum-free DMEM (DMEM) was added up to 1.5 ml. Both reagents in Tube1 and Tube2 were properly mixed respectively. Then Tube1 was vortexed and PEI in Tube 2 was added into Tubeldrop by drop to obtain a plasmid-PEI mixture solution, and the solution was kept for 30 minutes at room temperature; The mixture was added into 293T culture for transfection without suspending cells. Transfected cells were cultured for 24 hours in a 37° C./CO₂ incubator. Then the cell culture media was discarded and 20 ml of fresh 10% FBS-DMEM was supplemented. Finally, sodium butyrate was added until the final concentration of sodium butyrate was 10 mM. The cells were cultured for further 48 hours in an incubator with 5% CO₂ at 37° C.

3.) The cell culture supernatant was collected by centrifuging for 15 minutes at 4000 g/min, and the supernatant was filtered through a 0.45-micron filter to obtain 1×10\hat{ }6 TU/ml of virus solution. To origin virus solution adding 1/3 volume of a 40% PEG solution (g/g) and standing overnight at the temperature of 4° Cafter mixing. On the following day the precipitate of viruses is centrifuged at 1800 g for 45 minutes at 4° C. and the supernatant was discarded. The virus precipitate was re-suspended with 1/10 of original volume of culture media (X-VIVO15 for T cells, MEM-Alpha for NK92 cells) to obtain a 10-fold concentrated virus suspension;

4.) 3×10\hat{ }5 NK92 (ATCC, CRL -2407) and 1 ×10\hat{ }6 T cells (T cells from a healthy blood donor, separated by gradient centrifugation, stimulated and cultured by using OKT3) were infected respectively with 1×10\hat{ }7 virus particles, and 8 μg/ml polybrene (Sigma and H9268-5 G) was added. Infected cells were transferred to one well of a 24-well plate, and centrifugedat 1500R for 45 minutes at 32° C.The plate was incubated at 37° C., 5%CO₂ for 3 hours and the media was exchanged with media containing 100U/ml of IL-2 (for T cells 100ng/m1 of OKT3 was required) and the plate was incubated with the same conditions as above to obtain RaceCar-1-NK92 and RaceCar-1-T for the following experiments.

5.) With the same methods described as above onlypFUGW-RaceCar-1 vector was replaced with one among pFUGW-RaceCar-2 to pFUGW-RaceCar-14 respectively to obtain corresponding RaceCar-2-NK92, RaceCar-3-NK92, RaceCar-4-NK92,RaceCar-5-NK92, RaceCar-6-NK92, RaceCar-7-NK92, RaceCar-8-NK92, RaceCar-9-NK92, RaceCar-10-NK92,RaceCar-11-NK92,RaceCar-12-NK92,RaceCar-13-NK92,RaceCar-14-NK92 and RaceCar-2-T, RaceCar-3-T, RaceCar-4-T, RaceCar-5-T, RaceCar-6-T, RaceCar-7-T, RaceCar-8-T, RaceCar-9-T, RaceCar-10-T, RaceCar-11-T, RaceCar-12-T, RaceCar-13-T and RaceCar-14-T cells.

### EXAMPLE 5

### Expression of RaceCar in NK92 and Verification of Targeted Killing of RaceCar-NK92

1.) Target cell 3m-CD19-luc was prepared by integrating transgenes of CD19 (SEQ. ID. NO. 36) and luciferase (SEQ. ID. NO. 37) to the genome of Malme-3M cell (ATCC, HTB-64). Experimental groups were divided as follows: 1.) RaceCar-1-NK92 prepared by using example 4 serves as effector cells; 2.) RaceCar-2-NK92 prepared by using example 4 serves as effector cells; 3.) RaceCar-3-NK92 prepared by using example 4 serves as effector cells; 4.) RaceCar-4-NK92 prepared by using example 4servesas effector cells; 5.) RaceCar-5-NK92 prepared by using example 4 is used as effector cells; 6.) NK92 used as effector cells; 7.) RaceCar-1-NK92 used as effector cells; 8.) RaceCar-2-NK92 as effector cells. The cytotoxicity assay was carried out after an anti-CD19 antibody (biotin labelled mouse anti-Human CD19 antibody, Beijing Biodragon Immune Technologies, Beijing,BDLS-1968-100) was added in group 7 and 8. The target cell only used as negative control group.

2.) The effector cells were mixed with 1×10\hat{ }4 target cells in 100 μl in a 96-well plate at a ratio of 10:1, 5:1, 1:1, and incubated at 37° C. , in 5% CO₂ incubator for 24 h. 50 pi of 1% Triton lysate plus 1μl of substrate (mixture of 300 μg/mL Luc solution and 2 mg/mL ATP solution in a volume ratio of 3:1) was added into each well, the cells were lysed for 5 min, and luciferase fluorescence intensity was detected. The killing efficiency was calculated as follows={(negative control fluorescence) Value−experimental group fluorescence value}/negative control fluorescence value*100%.

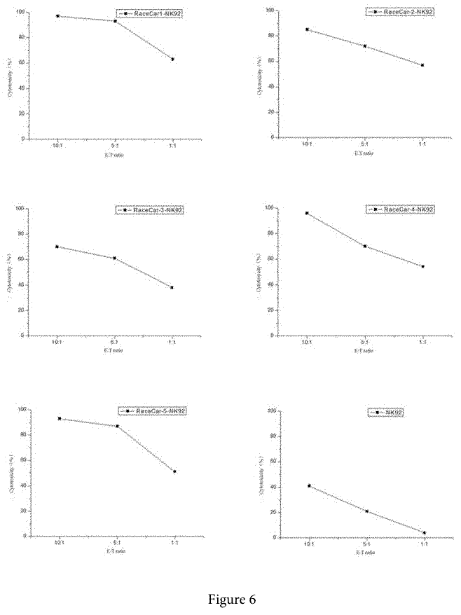

3.) The results are shown in FIG. 6. It is shown that RaceCar-1-NK92, RaceCar-2-NK92, RaceCar-3-NK92, RaceCar-4-NK92, and RaceCar-5-NK92 can kill 3m-CD19-luc cells more efficiently than that of NK92, which demonstrated that the multi-target chimeric antigen receptor RaceCar-1 targeting CD19 and PD-Ll is expressed in NK92 cells and has biological functions. In the experimental group to which the CD19 antibody was added, the killing ability of RaceCar-1-NK92 was decreased, while that of RaceCar-2-NK92 was almost unchanged, because anti-CD19 antibody to the target cells blocked the binding of the CAR on effector cells to the target cells, thereby inhibiting killing. The killing ability of RaceCar-1-NK92 against target cells demonstrated that RaceCar-NK92 is specific for the killing of 3m-CD19-luc cells.

### EXAMPLE 6

### Expression of RaceCar in T Cells and Comparison of the Targeted Killing Between RaceCar-T and Car-T

1.) The cell line k562-cd19-luc was obtained by incorporating nucleic acid sequences encoding CD19 antigen (SEQ. ID. NO. 36) and luciferase (SEQ. ID. NO. 37) into the genome of k562 cell line (ATCC) to express CD19 antigen and luciferase and used as a target cell. The effector cells used were RaceCar-1-T, RaceCar-4-T, and RaceCar-5-T obtained in Example 4, and the control effector cells used were Car-T cells (antiCDl9ScFv-CD8TM-41BB-CD3ζ chimeric antigen receptor, SEQ. ID. NO. 38) obtained by virus transfecting into T cells according to the above to express a conventional antiCD19ScFv-CD8TM-41BB-CD3ζ chimeric antigen receptor, target cells only without adding effector cells were used as a negative control for cell killing experiments.

2.) k562-cd19-luc cells were plated at 1×10\hat{ }4 cells/well, and effector cells were added at a ratio of effector cells to target cells of 5:1, 10:1, and 20:1, respectively. After 24 h, 50 μl of 1% Triton lysate +1μl substrate (mixture of 300 μg/mL Luc aqueous solution and 2 mg/mL ATP aqueous solution at a volume ratio of 3:1) were added to the cells, and lysed for 5 min. The luciferase luminescence was detected by a microplate reader to calculate the killing. Efficiency={(negative control fluorescence value−experimental group fluorescence value)/negative control fluorescence value}×100%.

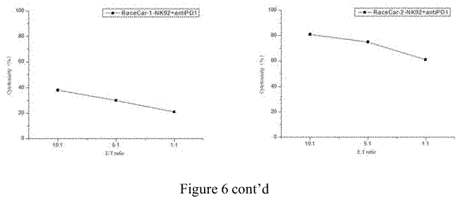

The results are shown in FIG. 7. In the figure, the abscissa is the ratio of effector cells to target cells, and the ordinate is the killing efficiency. It can be seen that RaceCar-1-T, RaceCar-4-T, and RaceCar-5-T cells can kill k562-cd19-luc cells, which demonstrates that RaceCar-1, RaceCar-4-T, RaceCar-5-T are expressed in T cells and have biological functions; and the killing ability of RaceCar-T cells is higher than that of common Car-T cells.

### EXAMPLE 7

### Cell Killing of Different Types of RaceCar-T cells and RaceCar-NK92 Cells Targeting to MHC/WT1

1.) The cell line BV173-luc was obtained by incorporating a nucleic acid sequence encoding a luciferase (SEQ. ID. NO._37) into the genome of BV173 cell line (DSMZ, ACC20) expressing a GP100/MHC molecular complex on the surface and as a target cell. The effector cells used were RaceCar-6-NK92, RaceCar-7-NK92, RaceCar-8-NK92, RaceCar-9-NK92, RaceCar-10-NK92, RaceCar-11-NK92, and RaceCar-12-NK92 obtained in Example 4. RaceCar-13-NK92, RaceCar-14-NK92 and RaceCar-6-T, RaceCar-7-T, RaceCar-8-T, RaceCar-9-T, RaceCar-10-T, RaceCar-11-T, RaceCar-12-T, RaceCar-13-T, RaceCar-14-T, control effector cells were uninfected NK92 cells or T cells, and cell killing experiments were performed without adding effector cells as a negative control.

2.) BV173-luc cells were plated at 1×10\hat{ }4 cells/well, and the effector cells were added to the target cells at a ratio of 5:1. After 24 h, 50 μl of 1% Triton lysate +1 μl substrate (300 μl g/mL Luciferin solution and 2 mg/mL ATP were mixed at a volume ratio of 3:1) were added to the cells, and lysed for 5 min. The luciferase luminescence was detected by a microplate reader. Killing efficiency={(negative control fluorescence value−experimental group fluorescence value)/negative control fluorescence value}×100%.

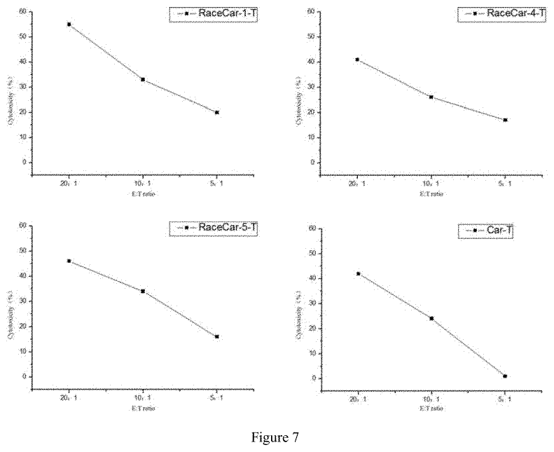

The results are shown in FIG. 8. In the figure, the abscissa is different effector cells, and the ordinate is the killing efficiency. It can be seen that both RaceCar-NK92 and RaceCar-T cells can kill BV173-luc cells and the efficiency is higher than that by uninfected T cells or NK92 cells, which demonstrates that RaceCar-6, RaceCar-7, RaceCar-8, RaceCar-9, RaceCar-10, RaceCar-11, RaceCar-12, RaceCar-13, and RaceCar-14 can be expressed in NK92 cells or T cells and have the biological function of mediating effector cells to kill target cells.

### EXAMPLE 8

### Effect of RaceCar on the Proliferation of Immune Cells

1.) 1×10\hat{ }6 of RaceCar-1-T and RaceCar-2-T cells obtained in Example 4 were placed in 2.5 ml of X-VIVO medium, and 1×10\hat{ }6 T cells were placed as a negative control in 2.5 ml of X-VIVO medium. Another 1×10\hat{ }6 T of cells was placed as a positive control in 2.5 mL of X-VIVO medium supplemented with IL-2 at a final concentration of 100 U/ml. The four groups of cells were incubated at 37° C., 5% CO₂.

2.) The working solution was prepared by dissolving CSFE dye into DMSO as 5 μmol/L solution. At the beginning of the culture and the 5th day of culture, 1×10\hat{ }4 cells were removed from the four groups of cells and the CSFE dye was added into each group of cells according to the ratio of CSFE working solution: medium volume ratio 1:2000 and incubated at 37° C. for 30 minutes.

3.) The supernatant was discarded by centrifugation at 1000 g for 5 minutes, and the cells were re-suspended using an equal volume of X-VIVO medium. This procedure was repeated twice.

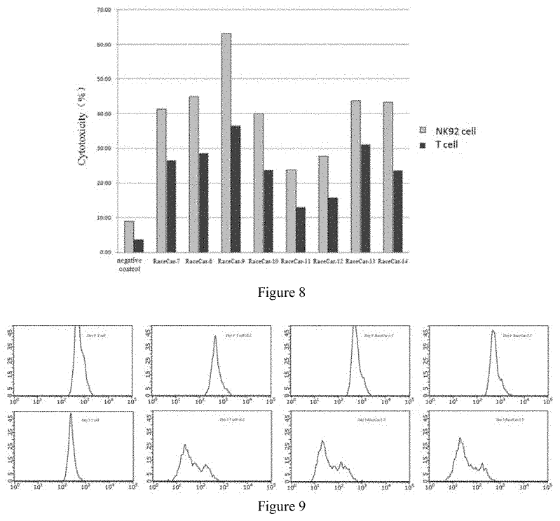

4.) The cells stained with CSFE would have green fluorescence and the intensity of green fluorescence would reduce as the cells divide. The cells of different generations show different fluorescence signals. The FACS spectrum is used to analyze the fluorescence of cells. In FIG. 9, it can be seen that at day 0, the fluorescence signals of the four groups of cells were basically the same. The T cell group was the negative control group. The fluorescence intensity of the flow detection on the 5th day was basically unchanged compared with that on day 0. T cell+IL2 was the positive control group and the cells showed different fluorescence signals between 5th day and the day 0, which confirmed that the cells proliferated during 5 days of culture. As shown on the figure, the distribution of the fluorescence signals of RaceCar-1-T and RaceCar-2-T was similar to that of the positive control group with IL-2 on the 5th day, demonstrating that RaceCar-1-T and RaceCar-2-T can also proliferate normally in the medium without IL2. The overall results prove that RaceCar has an ability to promote the proliferation of immune cells such as T cells.

### INDUSTRIAL APPLICATION

The experiments of the present invention prove that the multi-target chimeric antigen receptor of the present invention can bind to different antigens through its two antigen binding domains and mediate specific cell killing, can also improve the accuracy of therapeutic targeting, and can circumvent the relapse of the disease caused by down-regulation of single target expression; The multi-target chimeric antigen receptor of the present invention can bind to different antigens through its two antigen-binding domains, and can block the immune suppression signal and improve the ability to kill tumors if one of the antigen-binding domains binds to the antigen related to immune checkpoint proteins. The multi-target chimeric antigen receptor of the present invention introduces a cytokine and cytokine receptor complex, which can produce the function of cytokines, for example, the introduction of a complex of IL-15 and IL-15Rα-sushi stimulates the proliferation of T and NK cells, exempting them from cytokine-dependent activation.

What is claimed is:

1. A multi-target chimeric antigen receptor is composed of a main peptide chain and a co-peptide chain;The main peptide chain consists an antigen binding domain A, a co-peptide chain connecting domain B, a transmembrane domain C and an intracellular signalling structure domain D; the co-peptide chain includes a main peptide chain-linking domain F; the antigen-binding domain A is a polypeptide having an abilitytobind to an antigen; the co-peptide chain-linking domain B can bind to the main-peptide chain-linking domain F with each other; The co-peptide chain domain B and the main peptide chain domain F are cytokines and full length of corresponding cytokine receptors or cytokines and corresponding cytokine receptors fragment that can bind to each other; The transmembrane domain C is the transmembrane region of any membrane-bound protein or the transmembrane region of a transmembrane protein; the intracellular signalling domain D comprises a primary signalling region.

2. The multi-target chimeric antigen receptor according to claim 1, wherein: the co-peptide chain further comprises an antigen-binding domain E; and the antigen-binding domain E is a polypeptide withan antigen-binding function; The antigen-binding domain E and the antigen binding domain A can be the same or different.

3. (canceled)

4. The multi-target chimeric antigen receptor according to claim 1, wherein the polypeptide with an antigen-binding abilityis an antibody capable of binding to an antigen, a ligand capable of binding to an antigen, or a receptor with an ability to bind to an antigen.

5. The multi-target chimeric antigen receptor according to claim 4, wherein the antibody capable of binding to anantigen is a full length antibody, Fab of an antibody, Fc of an antibody, ascFv, a VHH, VH of an antibody, A full-length polypeptide of VL of anantibody or a partial fragment of VL of anantibody; the antigen-binding ligand or the antigen-binding receptor is a full-length polypeptide or a partial fragment of aligand or a receptor.

6. The multi-target chimeric antigen receptor according to claim 2, wherein the antigen bound by the antigen binding domain A orthe antigen binding domain E is either a cell surface antigen or an MHC moleculewith a peptide.

7. The multi-target chimeric antigen receptor according to claim 6, wherein the antigen is a cancer-associated antigen; or the antigen is an antigen related tobrain cancer, bladder cancer, breast cancer, cervical cancer, colorectal cancer, liver cancer, kidney cancer, lymphoma, leukaemia, lung cancer, melanoma, metastatic melanoma, mesothelioma, neuroblastoma, ovarian cancer, prostate cancer, pancreatic cancer, kidney cancer, skin cancer, thymoma, sarcoma, non-Hodgkin's lymphoma, Hodgkin's lymphoma, uterine cancer; or any combination thereof

8. The multi-target chimeric antigen receptor according to claim 7, wherein the antigen bound bythe antigen-binding domain A and the antigen-binding domain E is as follows: CD123, CD19, CD20, CD22, CD37, ROR1, mesothelin, CD33/IL3Ra, c-Met, BCMA, PSMA, EGFRvIII, GD-2, NY-ESO-1, MAGEA3, β-human chorionic gonadotropin, AFP, RAGE-1, MN-CA IX, human telomerase reverse transcriptase, RU1, RU2(AS), hsp70-2, M-CSF, PSA, PAP, LAGE-1a, p53, Prostein, PSMA, Her2/neu, Telomerase, PCTA-1, MAGE, ELF2M, IGF-I, IGF-II, IGF-I receptor, BCR-ABL, E2A-PRL, H4-RET, IGH-IGK, MYL-RAR, GP100, Martl, TSP-180, MAGE-4, MAGE-5, MAGE-6, RAGE, p185erbB2, p180erbB-3, c-met, nm-23H1, TAG-72, CA 19-9, CA 72-4, CAM 17.1, NuMa , K-ras, β-catenin, CDK4, Mum-1, p15, p16, 43-9F, 5T4, 791Tgp72, β-HCG BCA225, BTAA, CA 125, CA 15-3\CA 27.29\BCAA, CA 195, CA 242, CA-50, WT1, CD68, FGF-5, G250, EpCAM, MA-50, MG7-Ag, MOV 18, NB/70K, RCAS1, SDCCAG16, TA-90, TAAL6, T AG72, TLP, p53, Ras, TPS, Epstein Barr virus antigen EBVA and human papillomavirus (HPV) antigens E6 and E7 or any combination thereof; or The antigen bound by the antigen binding domain A and the antigen binding domain E is a complex of MHC and a short peptide of any oneantigen mentioned above.

9. The multi-target chimeric antigen receptor according to claim 8, wherein the antigen bound bythe antigen binding domain A and the antigen binding domain E is CD19, CD20, BCMA, CD22, CD33/IL3Ra , Her2, PDL1, NY-ESO-1, GP100, Marti, WT1 or any combination thereof; or the antigen bound by the antigen binding domain A and the antigen binding domain E is a complex of MHC and the short peptide of the aboveone of antigens.

10. The multi-target chimeric antigen receptor according to claim 1, wherein the cytokine and the corresponding cytokine receptor are cytokines and corresponding cytokines in the γc cytokine family and its receptors.

11. The multi-target chimeric antigen receptor according to claim 10, wherein the cytokine and the corresponding cytokine receptor in theyc cytokine family are IL15 and IL15Rα, IL4 and IL4Rα or IL2 and IL2Rα; Or the cytokine and the corresponding cytokine receptor in the γc cytokine family are polypeptides in which IL15 and IL15Rα, IL4 and IL4Rα, IL2 or IL2Rα have more than 75% homology.

12-13. (canceled)

14. The multi-target chimeric antigen receptor according to claim 1, wherein: in the multi-target chimeric antigen receptor, the antigen-binding domain A or E is a combination of one or both of an AntiCD19-ScFv, AntiMHC/GP100-VHH, AntiMHC/WT1-VH, AntiCD20-ScFv, AntiCD22-ScFv or PD1 extracellular region. The co-peptide linkage domain is IL15Rαsushi, IL4Rα-N-FN3, IL15 or IL4; The transmembrane domain is a transmembrane region of CD8 or a transmembrane region of CD28; The intracellular signalling domain is a CD3ζ signal transduction region, a polypeptide obtained by fusing a CD3ζ signalling region and a 4-1BB signalling region, or a polypeptide obtained by fusing a CD3ζ signalling region to a CD28 signaling region; The main peptide chain connectingdomain is IL15, IL4, IL15Rαsushi or IL4Rα Or N-FN3.

15-27. (canceled)

28. The multi-target chimeric antigen receptor according to claims 14, wherein: the multi-target chimeric antigen receptor is selected from the following:
(1) The antigen-binding domain A is AntiCD19-ScFv,the co-peptide linkage domain is IL15Rαsushi,the transmembrane domain is a transmembrane region of CD8,the intracellular signalling domain is a polypeptide obtained by fusing a CD3ζ signalling region and a 4-1BB(CD137) signalling region,the antigen binding domain E is an extracellular region of PD1, the main peptide chain connecting domain is IL15;
(2) The antigen-binding domain A is AntiMHC/GP100-VHH, the co-peptide linkage domain is IL15Rαsushi,the transmembrane domain is a transmembrane region of CD8,the intracellular signalling domain is a polypeptide obtained by fusing a CD3ζ signalling region and a 4-1BB(CD137) signalling region,the antigen binding domain E is an extracellular region of PD1, the main peptide chain connecting domain is IL15;
(3) The antigen-binding domain A is AntiMHC/GP100-VHH,the co-peptide linkage domain is IL15Rαsushi,the transmembrane domain is a transmembrane region of CD8,the intracellular signalling domain is a polypeptide obtained by fusing a CD3ζ signalling region and a 4-1BB(CD137) signalling region ,the antigen binding domain E is AntiMHC/Martl-VHH, the main peptide chain connecting domain is IL15;
(4) The antigen-binding domain A is AntiCD19-ScFv,the co-peptide linkage domain is IL15Rαsushi, the transmembrane domain is a transmembrane region of CD8,the intracellular signalling domain is a polypeptide obtained by fusing a CD3ζ signalling region and a 4-1BB(CD137) signalling region ,the antigen binding domain E is AntiCD20-ScFv, the main peptide chain connecting domain is IL15;
(5) The antigen-binding domain A is AntiCD19-ScFv,the co-peptide linkage domain is IL15Rαsushi,the transmembrane domain is a transmembrane region of CD8,the intracellular signalling domain is a polypeptide obtained by fusing a CD3ζ signalling region and a 4-1BB(CD137) signalling region ,the antigen binding domain E is AntiCD22-ScFv, the main peptide chain connecting domain is IL15;
(6) The antigen-binding domain A is AntiMHC/WT1-VH,the co-peptide linkage domain is IL15Rαsushi,the transmembrane domain is a transmembrane region of CD8,the intracellular signalling domain is a polypeptide obtained by fusing a CD3ζ signalling region and a 4-1BB(CD137) signalling region ,the antigen binding domain E isan extracellular region of PD1, the main peptide chain connecting domain is IL15;
(7) The antigen-binding domain A is AntiMHC/WT1-VH,the co-peptide linkage domain is IL15Rαsushi,the transmembrane domain is a transmembrane region of CD8,the intracellular signalling domain is a polypeptide obtained by fusing a CD3ζ signalling region and a CD28 signalling region ,the antigen binding domain E isan extracellular region of PD1, the main peptide chain connecting domain is IL15;
(8) The antigen-binding domain A is AntiMHC/WT1-VH,the co-peptide linkage domain is IL4Rα-N-FN3,the transmembrane domain is a transmembrane region of CD8,the intracellular signalling domain is a polypeptide obtained by fusing a CD3ζ signalling region and a 4-1BB (CD137) signalling region ,the antigen binding domain E is an extracellular region of PD1, the main peptide chain connecting domain is IL4;
(9) The antigen-binding domain A is AntiMHC/WT1-VH, the co-peptide linkage domain is IL15Rαsushi,the transmembrane domain is a transmembrane region of CD28, the intracellular signalling domain is a polypeptide obtained by fusing a CD3ζ signalling region and a 4-1BB (CD137) signalling region ,the antigen binding domain E isan extracellular region of PD1, the main peptide chain connecting domain is IL15;
(10) The antigen-binding domain A is an extracellular region of PD1,the co-peptide linkage domain is IL15Rαsushi,the transmembrane domain is a transmembrane region of CD8,the intracellular signalling domain is a polypeptide obtained by fusing a CD3ζ signalling region and a 4-1BB (CD137) signalling region ,the antigen binding domain E isAntiMHC/WT1-VH, the main peptide chain connecting domain is IL15;
(11) The antigen-binding domain A is AntiMHC/WT1-VH,the co-peptide linkage domain is IL15Rαsushi,the transmembrane domain is a transmembrane region of CD8,the intracellular signalling domain is a polypeptide obtained by fusing a CD3ζ signalling region and a 4-1BB (CD137) signalling region , the main peptide chain connecting domain is IL15;
(12) The antigen-binding domain A is AntiMHC/WT1-VH,the co-peptide linkage domain is IL15,the transmembrane domain is a transmembrane region of CD8,the intracellular signalling domain is a polypeptide obtained by fusing a CD3ζ signalling region and a 4-1BB (CD137) signalling region , the main peptide chain connecting domain is IL15 Rαsushi;
(13) The antigen-binding domain A is AntiMHC/WT1-VH,the co-peptide linkage domain is IL15,the transmembrane domain is a transmembrane region of CD8,the intracellular signalling domain is a polypeptide obtained by fusing a CD3ζ signalling region and a 4-1BB (CD137) signalling region ,the antigen binding domain E isan extracellular region of PD1, the main peptide chain connecting domain is IL15Rαsushi;
(14) The antigen-binding domain A is AntiMHC/WT1-VH, the co-peptide linkage domain isIL15Rαsushi,the transmembrane domain is a transmembrane region of CD8,the intracellular signalling domain is a polypeptide obtained by fusing a CD3ζ signalling region and a 4-1BB (CD137) signalling region ,the antigen binding domain E isAntiMHC/WT1-VH, the main peptide chain connecting domain is IL15.

29. The recombinant vector, expression cassette, recombinant microbe strain, cell or recombinant virus of the nucleic acid molecule encoding the multi-target chimeric antigen receptor according to claim 1, the nucleic acid molecule encoding a multi-target chimeric antigen receptoris composed to a nucleic acid molecule of primary peptide chain or a co-peptide chain.

30. The cell according to claim 29, wherein the cell is a prokaryotic cell, a yeast cell or a mammalian cell;

31. The cell according to claim 30, wherein the mammalian cell is specifically a human cell.

32. The cell according to claim 31, wherein the human cell is specifically an immune cell.

33. The cell according to claim 32, wherein Alternatively, the immune cell is specifically a T cell or an NK cell.

34. A method for promoting immune cell proliferation, including following procedure:
expressing the multi-target chimeric antigen receptor according to claim 33 to the surface of immune cell to make immune cells can proliferate in the medium without IL2 or IL15.

35. The method according to claim 34, wherein the immune cell is specifically a T cell or NK cell.


SEQUENCE LISTING (54 sequences)

SEQ ID NO: 1 - protein, 245 aa, Artificial sequence
  Synthesized
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 2 - protein, 150 aa, Artificial sequence [sequence omitted]

SEQ ID NO: 3 - protein, 114 aa, Artificial sequence
  Synthesized
NWVNVISDLKKIEDLIQSMHIDATLYTESDVHPSCKVTAMKCFLLELQVISLESGDASIHDTVENLIILANNSLSSNGNVTESGCKECEELEKKNIKEFLQSFVHIVQMFINTS

SEQ ID NO: 4 - protein, 72 aa, Artificial sequence
  Synthesized
ITCPPPMSVEHADIWVKSYSLYSRERYICNSGFKRKAGTSSLTECVLNKATNVAHWTTPSLKCIRDPALVHQ

SEQ ID NO: 5 - protein, 69 aa, Artificial sequence
  Synthesized
TTTPAPRPPTPAPTIASQPLSLRPEACRPAAGGAVHTRGLDFACDIYIWAPLAGTCGVLLLSLVITLYC

SEQ ID NO: 6 - protein, 112 aa, Artificial sequence
  Synthesized
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 41 aa, Artificial sequence
  Synthesized
RSKRSRLLHSDYMNMTPRRPGPTRKHYQPYAPPRDFAAYRS

SEQ ID NO: 8 - protein, 42 aa, Artificial sequence
  Synthesized
KRGRKKLLYIFKQPFMRPVQTTQEEDGCSCRFPEEEEGGCEL

SEQ ID NO: 9 - protein, 547 aa, Artificial sequence
  Synthesized
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSITCPPPMSVEHADIWVKSYSLYSRERYICNSGFKRKAGTSSLTECVLNKATNVAHWTTPSLKCIRDPALVHQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKL

SEQ ID NO: 10 - protein, 269 aa, Artificial sequence
  Synthesized
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSNWVNVISDLKKIEDLIQSMHIDATLYTESDVHPSCKVTAMKCFLLELQVISLESGDASIHDTVENLIILANNSLSSNGNVTESGCKECEELEKKNIKEFLQSFVHIVQMFINTS

SEQ ID NO: 11 - protein, 21 aa, Artificial sequence
  Synthesized
MALPVTALLLPLALLLHAARP

SEQ ID NO: 12 - protein, 22 aa, Artificial sequence
  Synthesized
GSGATNFSLLKQAGDVEENPGP

SEQ ID NO: 13 - DNA, 1698 bp, Artificial sequence
  Synthesized
atggccttaccagtgaccgccttgctcctgccgctggccttgctgctccacgccgccaggccggatatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggctccggcaagcccggatctggcgagggctccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtgagctcaggcggcggcggatctatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgaactcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcggcggaggggcaaggggcacgatggcctttaccagggtctcagtacagccaccaaggacacctacgacgcccttcacatgcaggccctgccccctcgc

SEQ ID NO: 14 - DNA, 879 bp, Artificial sequence
  Synthesized
ggaagcggcgccaccaacttctccctgctgaagcaagctggcgacgtggaagagaaccccggccccccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtgggcggaggaggctccaactgggtgaacgtcatctccgacctcaagaagatcgaggacctgatccagagcatgcacatcgacgccaccctgtataccgagagcgacgtgcacccctcctgtaaagtgaccgccatgaagtgcttcctgctggagctgcaggtgatcagcctggagagcggcgacgccagcatccatgacaccgtggagaacctgatcatcctggccaataacagcctgagctccaacggcaacgtgaccgagagcggctgcaaggaatgcgaggagctggagaagaagaacattaaggagttcctgcagagcttcgtccacatcgtgcagatgttcattaacacctcctaatga

SEQ ID NO: 15 - protein, 125 aa, Artificial sequence
  Synthesized
DIQLQASGGGLVQPGGSLRLSCAASGRTFNVDAMAWFRQTRGKEREFVAAISRSGGSTYYADSVKGRFSISKDNAKNTMYLQMNSLKPEDTAIYYCAAAIYWRGSYYTEGNYDYWGQRTQVTVSS

SEQ ID NO: 16 - protein, 117 aa, Artificial sequence
  Synthesized
DVQLQASGGGLVQAGDSLRLSCAASGRTFSTYNMGWFRQAPGKDREFVAAIMWSGGSTYYADSVKGRFTISKDNAKNTVYLQMNSLKPEDTAVYFCWSTDDYGVDSWGQGTQVTVSS

SEQ ID NO: 17 - protein, 243 aa, Artificial sequence
  Synthesized
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEI

SEQ ID NO: 18 - protein, 249 aa, Artificial sequence
  Synthesized
QVQLQQSGPGLVKPSQTLSLTCAISGDSVSSNSAAWNWIRQSTSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 19 - protein, 118 aa, Artificial sequence
  Synthesized
QLLESGGGLVQPGGSLRLSCAASGYRISSDDMSWVRQAPGKGLEWVSTIYETDGSTYYADSVKGRFTISRDNSKNTLYLQMNSLRAEDTAVYYCARANFYSEQPFQFWGQGTLVTVSS

SEQ ID NO: 20 - protein, 182 aa, Artificial sequence
  Synthesized
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQC

SEQ ID NO: 21 - protein, 129 aa, Artificial sequence
  Synthesized
HKCDITLQEIIKTLNSLTEQKTLCTELTVTDIFAASKNTTEKETFCRAATVLRQFYSHHEKDTRCLGATAQQFHRHKQLIRLLKRLDRNLWGLAGLNSCPVKEANQSTLENFLERLKTIMREKYSKCSS

SEQ ID NO: 22 - protein, 27 aa, Artificial sequence
  Synthesized
FWVLVVVGGVLACYSLLVTVAFIIFWV

SEQ ID NO: 23 - protein, 425 aa, Artificial sequence
  Synthesized
DIQLQASGGGLVQPGGSLRLSCAASGRTFNVDAMAWFRQTRGKEREFVAAISRSGGSTYYADSVKGRFSISKDNAKNTMYLQMNSLKPEDTAIYYCAAAIYWRGSYYTEGNYDYWGQRTQVTVSSGGGGSITCPPPMSVEHADIWVKSYSLYSRERYICNSGFKRKAGTSSLTECVLNKATNVAHWTTPSLKCIRDPALVHQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 418 aa, Artificial sequence
  Synthesized
QLLESGGGLVQPGGSLRLSCAASGYRISSDDMSWVRQAPGKGLEWVSTIYETDGSTYYADSVKGRFTISRDNSKNTLYLQMNSLRAEDTAVYYCARANFYSEQPFQFWGQGTLVTVSSGGGGSITCPPPMSVEHADIWVKSYSLYSRERYICNSGFKRKAGTSSLTECVLNKATNVAHWTTPSLKCIRDPALVHQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 376 aa, Artificial sequence
  Synthesized
QLLESGGGLVQPGGSLRLSCAASGYRISSDDMSWVRQAPGKGLEWVSTIYETDGSTYYADSVKGRFTISRDNSKNTLYLQMNSLRAEDTAVYYCARANFYSEQPFQFWGQGTLVTVSSGGGGSITCPPPMSVEHADIWVKSYSLYSRERYICNSGFKRKAGTSSLTECVLNKATNVAHWTTPSLKCIRDPALVHQFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 528 aa, Artificial sequence
  Synthesized
QLLESGGGLVQPGGSLRLSCAASGYRISSDDMSWVRQAPGKGLEWVSTIYETDGSTYYADSVKGRFTISRDNSKNTLYLQMNSLRAEDTAVYYCARANFYSEQPFQFWGQGTLVTVSSGGGGSMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 417 aa, Artificial sequence
  Synthesized
QLLESGGGLVQPGGSLRLSCAASGYRISSDDMSWVRQAPGKGLEWVSTIYETDGSTYYADSVKGRFTISRDNSKNTLYLQMNSLRAEDTAVYYCARANFYSEQPFQFWGQGTLVTVSSGGGGSITCPPPMSVEHADIWVKSYSLYSRERYICNSGFKRKAGTSSLTECVLNKATNVAHWTTPSLKCIRDPALVHQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 450 aa, Artificial sequence
  Synthesized
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSITCPPPMSVEHADIWVKSYSLYSRERYICNSGFKRKAGTSSLTECVLNKATNVAHWTTPSLKCIRDPALVHQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 460 aa, Artificial sequence
  Synthesized
QLLESGGGLVQPGGSLRLSCAASGYRISSDDMSWVRQAPGKGLEWVSTIYETDGSTYYADSVKGRFTISRDNSKNTLYLQMNSLRAEDTAVYYCARANFYSEQPFQFWGQGTLVTVSSGGGGSNWVNVISDLKKIEDLIQSMHIDATLYTESDVHPSCKVTAMKCFLLELQVISLESGDASIHDTVENLIILANNSLSSNGNVTESGCKECEELEKKNIKEFLQSFVHIVQMFINTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 236 aa, Artificial sequence
  Synthesized
DVQLQASGGGLVQAGDSLRLSCAASGRTFSTYNMGWFRQAPGKDREFVAAIMWSGGSTYYADSVKGRFTISKDNAKNTVYLQMNSLKPEDTAVYFCWSTDDYGVDSWGQGTQVTVSSGGGGSNWVNVISDLKKIEDLIQSMHIDATLYTESDVHPSCKVTAMKCFLLELQVISLESGDASIHDTVENLIILANNSLSSNGNVTESGCKECEELEKKNIKEFLQSFVHIVQMFINTS

SEQ ID NO: 31 - protein, 363 aa, Artificial sequence
  Synthesized
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSNWVNVISDLKKIEDLIQSMHIDATLYTESDVHPSCKVTAMKCFLLELQVISLESGDASIHDTVENLIILANNSLSSNGNVTESGCKECEELEKKNIKEFLQSFVHIVQMFINTS

SEQ ID NO: 32 - protein, 368 aa, Artificial sequence
  Synthesized
QVQLQQSGPGLVKPSQTLSLTCAISGDSVSSNSAAWNWIRQSTSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSNWVNVISDLKKIEDLIQSMHIDATLYTESDVHPSCKVTAMKCFLLELQVISLESGDASIHDTVENLIILANNSLSSNGNVTESGCKECEELEKKNIKEFLQSFVHIVQMFINTS

SEQ ID NO: 33 - protein, 237 aa, Artificial sequence
  Synthesized
QLLESGGGLVQPGGSLRLSCAASGYRISSDDMSWVRQAPGKGLEWVSTIYETDGSTYYADSVKGRFTISRDNSKNTLYLQMNSLRAEDTAVYYCARANFYSEQPFQFWGQGTLVTVSSGGGGSNWVNVISDLKKIEDLIQSMHIDATLYTESDVHPSCKVTAMKCFLLELQVISLESGDASIHDTVENLIILANNSLSSNGNVTESGCKECEELEKKNIKEFLQSFVHIVQMFINTS

SEQ ID NO: 34 - protein, 227 aa, Artificial sequence
  Synthesized
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSITCPPPMSVEHADIWVKSYSLYSRERYICNSGFKRKAGTSSLTECVLNKATNVAHWTTPSLKCIRDPALVHQ

SEQ ID NO: 35 - DNA, 1575 bp, Artificial sequence
  Synthesized
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaagggatccgagcccaaatcttgtgagaagacccacacatgcccaccgtgcccagcacctgaactcctggggggaccgtcagtgttcctcttccccccaaaacccaaggacaccctcatgatctcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacttgcctggtcaaaggcttctatcccagcgacatcgccgtggagtgggagagcaatgggcagccggagaacaactacaagaccacgcctcccgtgctggactccgacggctccttcttcctctactccaagctcaccgtggacaagagcaggtggcagcaggggaacgtgttctcatgctccgtgatgcatgaggctctgcacaaccactacacgcagaagagcctctccctgtctccgggtaaa

SEQ ID NO: 36 - DNA, 1671 bp, Artificial sequence
  Synthesized
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtga

SEQ ID NO: 37 - DNA, 1773 bp, Artificial sequence
  Synthesized
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattctcacggcttccctcccgaggtggaggagcaggccgccggcaccctgcccatgagctgcgcccaggagagcggcatggatagacaccctgctgcttgcgccagcgccaggatcaacgtc

SEQ ID NO: 38 - protein, 470 aa, Artificial sequence
  Synthesized
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKL

SEQ ID NO: 39 - protein, 284 aa, Artificial sequence
  Synthesized
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSHKCDITLQEIIKTLNSLTEQKTLCTELTVTDIFAASKNTTEKETFCRAATVLRQFYSHHEKDTRCLGATAQQFHRHKQLIRLLKRLDRNLWGLAGLNSCPVKEANQSTLENFLERLKTIMREKYSKCSS

SEQ ID NO: 40 - DNA, 1338 bp, Artificial sequence
  Synthesized
atggccttaccagtgaccgccttgctcctgccgctggccttgctgctccacgccgccaggccggatatccaactgcaagctagcgggggaggcttggtgcagcctggggggtctctgagactctcctgtgcagcctctggacgcaccttcaatgtcgatgctatggcttggttccgccagactcgcgggaaggagcgtgagtttgtagcagctattagccggagtggtggtagcacgtactatgcagactccgtgaagggccgattcagcatctccaaagacaacgccaaaaacacgatgtatctgcaaatgaacagcctcaaacctgaggacacggccatttattactgtgcagctgcaatctactggcgtggtagttactacactgaaggcaactatgactactggggccagaggacccaggtcaccgtgagctcaggcggcggcggatctatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgaactcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcggcggaggggcaaggggcacgatggcctttaccagggtctcagtacagccaccaaggacacctacgacgcccttcacatgcaggccctgccccctcgc

SEQ ID NO: 41 - DNA, 1320 bp, Artificial sequence
  Synthesized
atgtacaggatgcaactcctgtcttgcattgcactaagtcttgcacttgtcacgaattctcaggtgcagctgttggagtctgggggaggcttggtacagcctggggggtccctgcgtctctcctgtgcagcctccggatataggattagctctgacgatatgagctgggtccgccaggctccagggaagggtctagagtgggtatcaaccatttatgagacagacggtagcacatactacgcagactccgtgaagggccggttcaccatctcccgtgacaattccaagaacacgctgtatctgcaaatgaacagcctgcgtgccgaggacaccgcggtatattattgcgcgagagctaatttttatagtgagcagcccttccagttttggggtcagggaaccctggtcaccgtctcgagcggcggcggcggatctatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgaactcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcggcggaggggcaaggggcacgatggcctttaccagggtctcagtacagccaccaaggacacctacgacgcccttcacatgcaggccctgccccctcgc

SEQ ID NO: 42 - DNA, 1317 bp, Artificial sequence
  Synthesized
atgtacaggatgcaactcctgtcttgcattgcactaagtcttgcacttgtcacgaattctcaggtgcagctgttggagtctgggggaggcttggtacagcctggggggtccctgcgtctctcctgtgcagcctccggatataggattagctctgacgatatgagctgggtccgccaggctccagggaagggtctagagtgggtatcaaccatttatgagacagacggtagcacatactacgcagactccgtgaagggccggttcaccatctcccgtgacaattccaagaacacgctgtatctgcaaatgaacagcctgcgtgccgaggacaccgcggtatattattgcgcgagagctaatttttatagtgagcagcccttccagttttggggtcagggaaccctggtcaccgtctcgagcggcggcggcggatctatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgcccccccacgcgacttcgcagcctatcgctccagagtgaagttcagcaggagcgcagacgcccccgcgtacaagcagggccagaaccagctctataacgaactcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcggcggaggggcaaggggcacgatggcctttaccagggtctcagtacagccaccaaggacacctacgacgcccttcacatgcaggccctgccccctcgc

SEQ ID NO: 43 - DNA, 1650 bp, Artificial sequence
  Synthesized
atgtacaggatgcaactcctgtcttgcattgcactaagtcttgcacttgtcacgaattctcaggtgcagctgttggagtctgggggaggcttggtacagcctggggggtccctgcgtctctcctgtgcagcctccggatataggattagctctgacgatatgagctgggtccgccaggctccagggaagggtctagagtgggtatcaaccatttatgagacagacggtagcacatactacgcagactccgtgaagggccggttcaccatctcccgtgacaattccaagaacacgctgtatctgcaaatgaacagcctgcgtgccgaggacaccgcggtatattattgcgcgagagctaatttttatagtgagcagcccttccagttttggggtcagggaaccctggtcaccgtctcgagcggcggcggcggatctatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgaactcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcggcggaggggcaaggggcacgatggcctttaccagggtctcagtacagccaccaaggacacctacgacgcccttcacatgcaggccctgccccctcgc

SEQ ID NO: 44 - DNA, 1194 bp, Artificial sequence
  Synthesized
atgtacaggatgcaactcctgtcttgcattgcactaagtcttgcacttgtcacgaattctcaggtgcagctgttggagtctgggggaggcttggtacagcctggggggtccctgcgtctctcctgtgcagcctccggatataggattagctctgacgatatgagctgggtccgccaggctccagggaagggtctagagtgggtatcaaccatttatgagacagacggtagcacatactacgcagactccgtgaagggccggttcaccatctcccgtgacaattccaagaacacgctgtatctgcaaatgaacagcctgcgtgccgaggacaccgcggtatattattgcgcgagagctaatttttatagtgagcagcccttccagttttggggtcagggaaccctggtcaccgtctcgagcggcggcggcggatctatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaattttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgaactcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcggcggaggggcaaggggcacgatggcctttaccagggtctcagtacagccaccaaggacacctacgacgcccttcacatgcaggccctgccccctcgc

SEQ ID NO: 45 - DNA, 1410 bp, Artificial sequence
  Synthesized
atgtacaggatgcaactcctgtcttgcattgcactaagtcttgcacttgtcacgaattctccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtgggcggcggcggatctatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgaactcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcggcggaggggcaaggggcacgatggcctttaccagggtctcagtacagccaccaaggacacctacgacgcccttcacatgcaggccctgccccctcgc

SEQ ID NO: 46 - DNA, 1446 bp, Artificial sequence
  Synthesized
atgtacaggatgcaactcctgtcttgcattgcactaagtcttgcacttgtcacgaattctcaggtgcagctgttggagtctgggggaggcttggtacagcctggggggtccctgcgtctctcctgtgcagcctccggatataggattagctctgacgatatgagctgggtccgccaggctccagggaagggtctagagtgggtatcaaccatttatgagacagacggtagcacatactacgcagactccgtgaagggccggttcaccatctcccgtgacaattccaagaacacgctgtatctgcaaatgaacagcctgcgtgccgaggacaccgcggtatattattgcgcgagagctaatttttatagtgagcagcccttccagttttggggtcagggaaccctggtcaccgtctcgagcggcggcggcggatctaactgggtgaacgtcatctccgacctcaagaagatcgaggacctgatccagagcatgcacatcgacgccaccctgtataccgagagcgacgtgcacccctcctgtaaagtgaccgccatgaagtgcttcctgctggagctgcaggtgatcagcctggagagcggcgacgccagcatccatgacaccgtggagaacctgatcatcctggccaataacagcctgagctccaacggcaacgtgaccgagagcggctgcaaggaatgcgaggagctggagaagaagaacattaaggagttcctgcagagcttcgtccacatcgtgcagatgttcattaacacctccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgaactcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcggcggaggggcaaggggcacgatggcctttaccagggtctcagtacagccaccaaggacacctacgacgcccttcacatgcaggccctgccccctcgc

SEQ ID NO: 47 - DNA, 780 bp, Artificial sequence
  Synthesized
ggaagcggcgccaccaacttctccctgctgaagcaagctggcgacgtggaagagaaccccggccccgatgtgcagctgcaagctagcggaggcggcctggtgcaagctggagactccctgaggctgagctgcgccgctagcggaaggaccttcagcacctacaacatgggctggttcaggcaggcccccggaaaggacagggagttcgtggccgccattatgtggtccggcggcagcacctactatgccgacagcgtgaagggcaggttcacaatctccaaggacaacgccaagaacaccgtgtacctgcagatgaactccctgaagcccgaggatacagccgtgtacttctgctggagcaccgatgattacggagtcgacagctggggccagggaacccaggtgaccgtgagctccggcggaggaggctccaactgggtgaacgtcatctccgacctcaagaagatcgaggacctgatccagagcatgcacatcgacgccaccctgtataccgagagcgacgtgcacccctcctgtaaagtgaccgccatgaagtgcttcctgctggagctgcaggtgatcagcctggagagcggcgacgccagcatccatgacaccgtggagaacctgatcatcctggccaataacagcctgagctccaacggcaacgtgaccgagagcggctgcaaggaatgcgaggagctggagaagaagaacattaaggagttcctgcagagcttcgtccacatcgtgcagatgttcattaacacctcctaatga

SEQ ID NO: 48 - DNA, 1158 bp, Artificial sequence
  Synthesized
ggaagcggcgccaccaacttctccctgctgaagcaagctggcgacgtggaagagaaccccggccccgaggtgcagctggtggagagcggaggcggactggtgcagcctggcagatccctcagactgtcctgtgccgcctccggcttcaccttcaacgactacgccatgcactgggtgaggcaggcccccggaaaaggactggagtgggtgtccaccatcagctggaactccggatccatcggctacgccgactccgtgaagggcaggttcaccatctccagggataacgccaagaagtccctgtacctgcagatgaacagcctgagagccgaggacaccgccctgtactactgcgccaaggacatccagtacggaaactactactacggcatggacgtgtggggacaaggcaccaccgtgacagtgagcagcggcggaggaggatccggaggaggcggatccggaggaggaggatccgagatcgtgctgacccagagccctgctaccctgagcctgagccctggcgagagagccaccctgtcctgcagggctagccagtccgtgtccagctacctggcctggtaccagcagaagcccggccaagcccctaggctgctgatctacgacgccagcaatagggccacaggcatccccgccagatttagcggcagcggctccggaaccgacttcacactgaccatcagctccctggagcccgaagacttcgccgtgtattactgccagcagaggagcaactggcccatcaccttcggacagggcaccagactggagatcggcggaggaggctccaactgggtgaacgtcatctccgacctcaagaagatcgaggacctgatccagagcatgcacatcgacgccaccctgtataccgagagcgacgtgcacccctcctgtaaagtgaccgccatgaagtgcttcctgctggagctgcaggtgatcagcctggagagcggcgacgccagcatccatgacaccgtggagaacctgatcatcctggccaataacagcctgagctccaacggcaacgtgaccgagagcggctgcaaggaatgcgaggagctggagaagaagaacattaaggagttcctgcagagcttcgtccacatcgtgcagatgttcattaacacctcctaatga

SEQ ID NO: 49 - DNA, 1176 bp, Artificial sequence
  Synthesized
ggaagcggcgccaccaacttctccctgctgaagcaagctggcgacgtggaagagaaccccggcccccaggtgcagctgcaacagagcggccctggactggtgaagcccagccagacactgagcctcacctgcgccattagcggcgacagcgtgagctccaattccgccgcctggaattggatcagacagagcacaagcaggggcctggagtggctgggcaggacctactacaggagcaagtggtacaacgactacgccgtgtccgtgaagagcaggatcacaatcaaccccgacaccagcaagaaccagttcagcctgcagctcaacagcgtcacccccgaagacaccgccgtgtactactgcgccagagaggtgaccggcgacctggaggacgccttcgacatctggggccaaggcaccatggtgaccgtgagctccggctccacaagcggaagcggcaaacctggctccggcgagggttctaccaaaggcgacatccagatgacccagagcccttcctccctgagcgccagcgtgggcgatagagtgacaattacctgcagggccagccagaccatctggagctacctgaactggtaccagcagaggcccggaaaggcccccaacctgctgatctacgccgcttccagcctgcagagcggcgtgcctagcaggttctccggcagaggcagcggcaccgatttcaccctgacaatcagcagcctgcaggccgaagattttgccacctactactgccagcagagctacagcatcccccagaccttcggccagggcaccaaactggagatcaagggcggaggaggctccaactgggtgaacgtcatctccgacctcaagaagatcgaggacctgatccagagcatgcacatcgacgccaccctgtataccgagagcgacgtgcacccctcctgtaaagtgaccgccatgaagtgcttcctgctggagctgcaggtgatcagcctggagagcggcgacgccagcatccatgacaccgtggagaacctgatcatcctggccaataacagcctgagctccaacggcaacgtgaccgagagcggctgcaaggaatgcgaggagctggagaagaagaacattaaggagttcctgcagagcttcgtccacatcgtgcagatgttcattaacacctcctaatga

SEQ ID NO: 50 - DNA, 924 bp, Artificial sequence
  Synthesized
ggaagcggcgccaccaacttctccctgctgaagcaagctggcgacgtggaagagaaccccggccccccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtgggcggaggaggctcccacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgactcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctaatga

SEQ ID NO: 51 - DNA, 789 bp, Artificial sequence
  Synthesized
ggaagcggcgccaccaacttctccctgctgaagcaagctggcgacgtggaagagaaccccggcccccaggtgcagctgttggagtctgggggaggcttggtacagcctggggggtccctgcgtctctcctgtgcagcctccggatataggattagctctgacgatatgagctgggtccgccaggctccagggaagggtctagagtgggtatcaaccatttatgagacagacggtagcacatactacgcagactccgtgaagggccggttcaccatctcccgtgacaattccaagaacacgctgtatctgcaaatgaacagcctgcgtgccgaggacaccgcggtatattattgcgcgagagctaatttttatagtgagcagcccttccagttttggggtcagggaaccctggtcaccgtctcgagcggcggaggaggctccaactgggtgaacgtcatctccgacctcaagaagatcgaggacctgatccagagcatgcacatcgacgccaccctgtataccgagagcgacgtgcacccctcctgtaaagtgaccgccatgaagtgcttcctgctggagctgcaggtgatcagcctggagagcggcgacgccagcatccatgacaccgtggagaacctgatcatcctggccaataacagcctgagctccaacggcaacgtgaccgagagcggctgcaaggaatgcgaggagctggagaagaagaacattaaggagttcctgcagagcttcgtccacatcgtgcagatgttcattaacacctcctaatga

SEQ ID NO: 52 - DNA, 414 bp, Artificial sequence
  Synthesized
ggaagcggcgccaccaacttctccctgctgaagcaagctggcgacgtggaagagaaccccggccccaactgggtgaacgtcatctccgacctcaagaagatcgaggacctgatccagagcatgcacatcgacgccaccctgtataccgagagcgacgtgcacccctcctgtaaagtgaccgccatgaagtgcttcctgctggagctgcaggtgatcagcctggagagcggcgacgccagcatccatgacaccgtggagaacctgatcatcctggccaataacagcctgagctccaacggcaacgtgaccgagagcggctgcaaggaatgcgaggagctggagaagaagaacattaaggagttcctgcagagcttcgtccacatcgtgcagatgttcattaacacctcctaatga

SEQ ID NO: 53 - DNA, 288 bp, Artificial sequence
  Synthesized
ggaagcggcgccaccaacttctccctgctgaagcaagctggcgacgtggaagagaaccccggccccatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaataatga

SEQ ID NO: 54 - DNA, 753 bp, Artificial sequence
  Synthesized
ggaagcggcgccaccaacttctccctgctgaagcaagctggcgacgtggaagagaaccccggccccccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtgggcggaggaggctccatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaataatga